(19) United States
(12) Patent Application Publication
(10) Pub. No.: US 2019/0030375 A1
(43) Pub. Date: Jan. 31, 2019

(54) TRANSCRANIAL ULTRASOUND FOCUSING

(71) Applicant: Oron ZACHAR, Tel Aviv (IL)

(72) Inventor: Oron ZACHAR, Tel Aviv (IL)

(21) Appl. No.: 15/902,361

(22) Filed: Feb. 22, 2018

Related U.S. Application Data
(60) Provisional application No. 62/462,398, filed on Feb. 23, 2017.
(60) Provisional application No. 62/468,473, filed on Mar. 8, 2017.
(60) Provisional application No. 62/474,715, filed on Mar. 22, 2017.
(60) Provisional application No. 62/492,193, filed on Apr. 30, 2017.

(51) Int. Cl. A61N 7/00

(52) U.S. Cl. CPC A61N 7/00; A61N 2007/0078; A61N 2007/0021; A61N 2007/0095; A61N 2007/0082

(57) ABSTRACT

The invention introduces a system for focusing ultrasonic energy through intervening tissue into a target site within a target tissue region, includes a transducer emitter array, a transducer receiver array, a processor receiving echo signals from the receiver to determine correction factors for the transducer elements to compensate for refraction occurring due to intervening tissue. The correction factors may include phase correction factors, and the phases of excitation signals provided to the transducer elements may be adjusted based upon the phase correction factors to focus the ultrasonic energy to the tissue at the target site.

### FIELD OF INVENTION

The present invention relates, generally, to systems and methods for ultrasound focusing. In particular, various embodiments are directed to efficient methods of focusing a phased array of ultrasound transducer elements, using preparatory measurements to adjust the relative phases of the transducer elements.

### BACKGROUND OF THE INVENTION

Thermal ablation, as may be accomplished using focused ultrasound, has particular appeal for treating tissue within the brain and other tissue regions deep within the body, because it generally does not disturb intervening or surrounding healthy tissue. Focused ultrasound may also be attractive, because acoustic energy generally penetrates well through soft tissues, and ultrasonic energy, in particular, may be focused towards focal zones having a cross-section of only a few millimeters due to relatively short wavelengths (e.g., as small as 1.5 millimeters (mm) in cross-section at one Megahertz (1 MHz)). Thus, ultrasonic energy may be focused at a region deep within the body, such as a cancerous tumor or other diseased tissue, to ablate the diseased tissue without significantly damaging surrounding healthy tissue.

To focus ultrasonic energy towards a desired target, a piezoelectric transducer may be used that includes a plurality of transducer elements. A controller may provide drive signals to each of the transducer elements, thereby causing the transducer elements to transmit acoustic energy such that constructive interference occurs at a “focal zone”. The focal zone is typically defined as the region of intensity higher than half maximum, and is commonly characterized by a “peak width” in a given direction. The peak width may be anisotropic. In fact, most realized instrumental systems produce an elliptical shaped peak cross-section at half maximum. At the focal zone, sufficient acoustic energy may be delivered to generate the desired tissue activation (e.g., heating, necrosis, neural stimulation, etc . . . ) within the focal zone and for a sufficient period until tissue affects occurs. Preferably, tissue along the path through which the acoustic energy passes (“the pass zone”) outside the focal zone, is affected (e.g, heated) only minimally, if at all, thereby minimizing damaging tissue outside the focal zone.

Phased arrays of ultrasound transducers are well-known as a system for focusing ultrasound energy at target sites inside the body. Constructive and destructive interference of acoustic waves transmitted by multiple transducers can be used to deliver complex spatiotemporal patterns of acoustic waves. Generally, phased arrays use tens to hundreds or even thousands of ultrasound transducers distributed spatially on the surface of the body. For instance, a phased array placed on the head can be used to target an area deep in the brain. However, phased arrays have important limitations for delivering ultrasound transcranially for neuromodulation. Phased arrays use spatially distributed transducers, requiring a larger form factor. Moreover, large and generally unportable power and control components are required to manage the timing, intensity, phase, and other properties of the ultrasound waves transmitted by each of the transducers.

The prior art of ultrasound focusing onto tissue in general and brain tissue in particular is exemplified in USA patents U.S. Pat. No. 8,932,237, U.S. Pat. No. 5,329,930, U.S. Pat. No. 4,817,614, U.S. Pat. No. 8,088,067, U.S. Pat. No. 6,128,958, U.S. Pat. No. 7,611,462, U.S. Pat. No. 5,984,881, and references therein, the entire disclosures of which are hereby incorporated by reference.

Focused ultrasound (i.e., acoustic waves having a frequency greater than about 20 kilohertz) can be used to image or therapeutically treat internal body tissues within a patient. For example, ultrasonic waves may be used to ablate tumors, eliminating the need for the patient to undergo invasive surgery. For this purpose, a piezo-ceramic transducer is placed externally to the patient, but in close proximity to the tissue to be ablated (the “target”). The transducer converts an electronic drive signal into mechanical vibrations, resulting in the emission of acoustic waves (a process hereinafter referred to as “sonication”). The transducer may be shaped so that the waves converge in a focal zone. Alternatively or additionally, the transducer may be formed of a plurality of individually driven transducer elements whose phases (and, optionally, amplitudes) can each be controlled independently from one another and, thus, can be set so as to result in constructive interference of the individual acoustic waves in the focal zone. Such a “phased-array” transducer facilitates steering the focal zone to different locations by adjusting the relative phases between the transducers, and generally provides the higher a focus quality and resolution, the greater the number of transducer elements. Magnetic resonance imaging (MRI) may be utilized to visualize the focus and target in order to guide the ultrasound beam.

While the transducer is located external to the patient, it must be in direct contact and tightly coupled with a media that efficiently transmits the high frequency ultrasound waves. For example, the transducer can be positioned in a liquid bath that is capable of efficient transmission of the ultrasound waves. The patient's body must also be wetted and tightly coupled to the transmission media in order to ensure an optimal acoustic wave transmission path from the transducer to the focal zone.

While system parameters are generally fixed for a given transducer array, tissue homogeneity may vary significantly from patient to patient, and even between different tissue regions within the same patient. Tissue inhomogeneity may decrease intensity of the acoustic energy at the focal zone and may even move the location of the focal zone within the patient's body. Specifically, because the speed of sound differs in different types of tissue, as portions of a beam of acoustic energy travel along different paths towards the focal zone, they may experience a relative phase shift or time delay, which may change the intensity at the focal zone and/or move the location of the focal zone.

For example, the speed of sound through fat is approximately 1460 meters per second (m/s), while the speed of sound through muscle is approximately 1600 meters per second (m/s). The speed of sound through bone tissue is much faster, for example, approximately 3000 meters per second (m/s) for skull bone tissue. The speed of sound also varies in different organs. For example, the speed of sound in brain tissue is approximately 1570 meters per second (m/s), approximately 1555 meters per second (m/s) in the liver, and approximately 1565 meters per second (m/s) in the kidney.

The relative phases (alternatively relative “time shift”) at which the transducer elements need to be driven to result in a focus at the target location depend on the relative location and orientation of the transducer surface and the target, as well as on the dimensions and acoustic material properties (e.g., sound velocities) of the tissue or tissues between them (i.e., the “target tissue”). Thus, to the extent the geometry and acoustic material properties are known, the relative phases (and, optionally, amplitudes) can be calculated, as described, for example, in U.S. Pat. Nos. 6,612,988, 6,770,031, and 7,344,509, the entire disclosures of which are hereby incorporated by reference. In practice, however, knowledge of these parameters is often too incomplete or imprecise to enable high-quality focusing based on computations of the relative phases alone. For example, when ultrasound is focused into the brain to treat a tumor, the skull in the acoustic path may cause aberrations that are not readily ascertainable. In such situations, treatment is typically preceded by an auto-focusing procedure in which, iteratively, an ultrasound focus is generated at or near the target, the quality of the focus is measured (using, e.g., thermal imaging or acoustic radiation force imaging (ARFI)), and experimental feedback is used to adjust the phases of the transducer elements to achieve sufficient focus quality.

The auto-focusing procedure may thus take a substantial amount of time, which may render it impracticable or, at the least, inconvenient for a patient. While the effect of pre-therapeutic sonications may be minimized by employing an imaging technique that requires only low acoustic intensity (e.g., ARFI), it is generally desirable to limit the number of sonications prior to treatment. Accordingly, there is a need for more efficient ways of focusing a phased array of transducer element to create a high-quality ultrasound focus.

Another common technique for focusing ultrasound is by using a shaped lens with an acoustic velocity (i.e. speed of sound) that differs from adjoining air, tissue, or material to bend acoustic waves. Most standard ultrasound focusing lenses employ a single concave lens. However, a single concave lens focusing system for ultrasound has limitations, including limitations. Ultrasound lenses comprised of a single concave lens are limited with regard to the range of focal lengths that can be achieved with a lens of a particular cross sectional area. Short focal lengths cannot be achieved with smaller cross sectional areas appropriate for systems affixed to the head or skull. Neuromodulation of superficial brain regions with an appropriate transcranial ultrasound system would be advantageous due to the importance of such superficial brain regions (e.g. cerebral cortex) to sensory, motor, higher cognitive function, and other brain functions.

To affect brain function transcranial ultrasound neuromodulation requires appropriate ultrasound waveform parameters, including acoustic frequencies generally less than about 10 MHz, spatial-peak temporal-average intensity generally less than about 10 W/cm2 (e.g., between 0.5 and 10 W/cm2), and appropriate pulsing and other waveform characteristics to ensure that heating of a targeted brain region does not exceed about 2 degrees Celsius for more than about 5 seconds. Transcranial ultrasound neuromodulation induces neuromodulation primarily through vibrational or mechanical mechanisms. Noninvasive and nondestructive transcranial ultrasound neuromodulation is in contrast to other transcranial ultrasound based techniques that use a combination of parameters to disrupt, damage, destroy, or otherwise affect neuronal cell populations so that they do not function properly and/or cause heating to damage or ablate tissue.

As by in the article Lindsey (Lindsey B D, Smith S W. Refraction Correction in 3D Transcranial Ultrasound Imaging. Ultrasonic imaging. 2014; 36(1):35-54. doi:10.1177/0161734613510287), Image quality in transcranial ultrasound remains limited by the deleterious effects of the skull, including attenuation, aberration, refraction, and mode conversion. Effects of attenuation may be reduced by positioning the probe within an acoustic window in the temporal bone; however, this window is absent in 8% to 29% of individuals. Transmitting with large, lower frequency (^{˜}1 MHz) array probes may help reduce the dependence on acoustic window quality. The effects of aberration induced by spatially inhomogeneous layers having a different longitudinal wave velocity from that typically assumed by the ultrasound scanner (c=1540 m/s) may be addressed by one of the many techniques for phase aberration correction. Some of these aberration correction techniques include inherent correction for refraction using either ultrasound-based measurements in two-dimensional (2D) imaging or computed-tomography-based measurements in three-dimensional (3D) therapy though refraction correction in 3D imaging has not been demonstrated in vivo. Other techniques have modeled aberration as a distributed phenomenon rather than as a single spatially varying layer. Previously addressed anatomical sources of aberration include layers of bone in the skull (c≈2800 m/s, commonly within 15% variation due to difference in bone porosity and thickness) or layers of fat (c≈1450).

As discussed in the journal article by Ding et. al., Phys. Med. Biol. 60 (2015) 3975-3998, ultrasound penetration through the skull is better, with less energy deposition within the skull bone itself, at around 0.5 MHz compared with higher frequencies.

### SUMMARY OF THE INVENTION

There is a need for more efficient ways of focusing a phased array of transducer element to create a high-quality ultrasound focus without recourse to imaging model of the brain by MRI or Ultrasound.

As illustrated in FIG. 6A, when a transducer is activated, the relative time delay Tn (alternatively delineated “phase shift”) of drive signals delivered to each transducer emitter element E(n) may be adjusted. Assuming a specific uniform medium, characterized by a specific speed of sound, to the target focus 111, based upon the distance of the respective transducer element from the focal zone center 111 there is a well-defined path traversal time of the sound from each emitter to the target focus center 111. For example, for focusing into body tissue, an average speed of sound is used to approximate the speed at which the acoustic energy passes through tissue, e.g., 1540 meters per second (m/s), is used to predict the location of the focus center for a set {F0(n)} of relative time delays. In such uniform medium setting, the ultrasound focus is obtained with focal zone 119 having characteristics zone cross-section geometry and cross section area.

Yet, as illustrated in FIG. 6B, when ultrasound is focused into the brain, if using a uniform medium set {F0(n)}, the skull intervening bone-tissue intermediate-layer 150 in the acoustic path may cause aberrations that enlarge the focal zone 129 in comparison with the focal zone 119 width created in fully uniform medium seeting. The present invention provides a method and apparatus for improving (i.e., reducing the size of) the focal zone in such a situation.

Since a beam of acoustic energy has a relatively wide aperture where it enters the body, different parts of the acoustic energy, such as 123 and 124, may pass through different intervening tissue layer thickness between, which may shift the effective relative time delay of acoustic energy transmitted from respective transducer elements upon arrival to the focal zone. This phase shifting may decrease the constructive interference of the acoustic energy at the focal zone, or may even move the focal zone in an unpredictable manner. For example, an intervening skull bone layer thickness difference of 1.5 mm may introduce a phase shift of 180° at an ultrasonic frequency of one Megahertz (1 MHz), which would change desired constructive interference at the focal zone into destructive interference.

In preferred embodiments, ultrasound frequency lower than 1 MHz, such as between 100 KHz and 500 KHz, is used in order to reduce the aberration effect of non-bone tissue inhomogeneity in the brain.

As illustrated in FIG. 6C, the principle goal of the present invention is to drive the focusing transducer 110 with an adjusted signal delay set {F′(n)}={F0(n)+dFn}, such that where the ultrasound waves path pass through an intervening second-tissue intermediate-layer bounded by first and second boundary surfaces within otherwise same uniform medium 99 in the path from the transducer surface to the focus peak 131 with focal zone area 139. Due to the adjusted signal delay set {F′(n)}, an improved interference is creating the focus peak 139 with focal zone area 139 smaller than the focal zone 129 created when the focusing transducer 110 with an unadjusted signal delay set {F0(n)}.

The key problem is how to determine the adjustments set {dFn}. The present invention provides a method and system for determine the adjustments set {dFn} and thereby creating an improved focus when the focused ultrasound waves path pass through an intervening second-tissue intermediate-layer bounded by first and second boundary surfaces within otherwise approximately uniform medium 99 in the path from the transducer surface to the focus peak.

In general, as in prior art, we conceptualize the method procedure as composed of two major stages: (i) scanning procedure “PROC-1”, and (ii) adjusted irradiation application procedure “PROC-2”.

The scanning procedure PROC-1 can be sub-divided into two prominent sub-tasks: (a) physical scanning procedure “SCAN”, and (b) computational analysis “ANALYSIS” from which the key outcome is the determination of the adjustments set {dFn}.

The adjusted irradiation application procedure “PROC-2” can be sub-divided into two prominent sub-tasks: (a) Input parameters set-up, and (b) irradiation application process.

The adjusted irradiation application procedure “PROC-2” is similar to prior art. Given the adjustments set {dFn} from PROC-1, the system is activating the emitters array with the corrected set {F′n} of input parameters phases. The irradiation application process itself is determined by clinical goals (e.g., nerve stimulation, tissue ablation, etc. . . . ).

The innovation is primarily contained in the preparatory scanning process PROC-1 method and apparatus and from it the core outcome is the values of the adjustments set {dFn} of input parameters that is used to define the corrected set {F′n} of phases.

As known in prior art, there are multiple methods of determining the adjustments set {dFn} if there is a given geometry of the intermediate layer (e.g., skull bone layer). The problem is how to determine the skull properties and geometry non-invasively. For concreteness we define the intermediate layer geometry by knowledge of first boundary surface 151 shape function B1(x,y,z) and the second layer 152 shape function B2(x,y,z).

In prior art, for skull bone, the skull bone layer geometry is determined from MRI or CT imaging. Thus, in prior art the SCAN sub-task scanning procedure PROC-1 is done by MRI or CT scanning, from which various skull parameters are determined by supplemental external information. e.g., from multiple slices of MRI images a full 3D skull bone section shape is reconstructed. In addition, external information concerning speed of sound in the bone is supplemented to predict and determine the supposed time shifts created by the skull bone on ultrasound. i.e., the MRI or CT scanning is NOT by itself directly measuring ultrasound phase shift or time shift due to passage through the skull layer.

In prior art ANALYSIS sub-task, each phase shift correction dFn to be applied to individual emitter En is determined by going through a geometrical reconstruction of the intermediate layer shape and considering the particular path of the ultrasound from the emitter En to the intended focus location.

In contrast, the present invention: (i) uses ultrasound emitters array not only for the irradiation PROC-2 procedure, but also for the scanning PROC-1 procedure, thereby eliminating completely the need for non-ultrasound MRI or CT at any step of the full procedure (PROC-1 and PROC-2) method and apparatus; (ii) the ultrasound phase shift or time shift due to passage through the skull layer is directly measured by the SCAN procedure ultrasound scan, and (iii) the SCAN procedure ultrasound scan method in the present invention is different from what is commonly understood as “ultrasound scanning” in prior art. In particular, while the majority of the array elements {En} are activated simultaneously during the irradiation PROC-2 procedure (as in common ultrasound scanning), only individual emitter elements En (or a small fraction of the array elements, e.g., less than 20%) are activated simultaneously during the SCAN process of PROC-1.

In prior art scanning with ultrasound phase arrays, what is conventionally understood to be ultrasound scanning: (i) the array elements are operated to radiate simultaneously, (ii) a focus beam is used, and (iii) target area scanning is performed by steering the beam focus by way of modifying the relative phase shifts between simultaneously activated array elements. In order to find and trace the skull outer surface, prior art phased array scan is moving the focus over a volume within which the skull is assumed to be residing somewhere. In contrast, non of the above is conducted in the present invention ultrasound SCAN procedure.

The present invention SCAN sub-task is characterized by that: (i) the array ultrasound elements are operated serially in time, such that individual array element (or small groups of elements the majority of which consisting of less than 10% of the number array elements) are operated on after the other, and preferably after the previous element signal reflection have been measured; (ii) the ultrasound beam is not focused; (iii) target area scanning is performed by NOT by steering a beam focus, but instead by way of each individual array element (or small group of elements) measuring the small section of the intermediate layer (e.g., skull bone) closest to it.

In addition, in preferred embodiments of the present invention ANALYSIS sub-task, each phase shift correction dFn to be applied to individual emitter En is determined directly from the associated individual emitter En SCAN step, without going through a geometrical reconstruction of the intermediate layer shape and without considering the particular path of the ultrasound from the emitter to the intended focus.

In preferred embodiments,

dFn=(V2/V1−1)*Tn/2

i.e.,

F′(n)=F0(n)+(V2/V1−1)*Tn/2,

where V1 is an assumed average speed of sound in the interior brain tissue (preferably within 10% accuracy), and V2 is an assumed average speed of sound in the intermediate layer (e.g., skull bone), preferably within 20% accuracy (or better). The time Tn is determined from the time difference between the reflected signals 171 from the intermediate layer first surface 151 and the reflected signals 172 from the intermediate layer second surface 152.

In other preferred embodiments, ANALYSIS sub-task is more conventionally performed, such that each phase shift correction dFn to be applied to individual emitter En is computed by going through a geometrical reconstruction of the intermediate layer shape and considering the particular path of the ultrasound from the emitter En to the intended focus location. Yet, in the present invention the geometrical reconstruction of the intermediate layer shape and thickness are determined in a novel way.

For example, as illustrated in FIG. 7, in a SCAN step, individual emitter 141n is emitting a signal while all other emitters (or at least nearby emitters) are not activated. The reflected echo signal 171 from the intermediate layer first surface 151 and the reflected echo signal 172 from the intermediate layer second surface 152 are each detected at the same receiver sensor 161n (in practice the physical receiver sensor 161n can be the same as the emitter source 141n), and the time difference Tn between them is determined (e.g., the time difference between the maximum peaks of the received echo signals). Similarly, such SCAN steps are serially performed in time on other emitters (preferably most of the emitters, or all of the emitters), thus generating the echo time difference set {Tn}.

We interpret and associate each measured Tn as an estimation of the local time difference of ultrasound to so twice across the local skull bone thickness nearest to the emitter element En.

In preferred embodiments, the adjustments elements dFn forming the adjustment set {dFn} are each a function of Tn, V1 and V2. Preferably, as noted before,

dFn=(V2/V1−1)*Tn/2.

We interpret and associate an estimated local skull bone thickness Wn, where

Wn=V2*Tn/2,

as the local skull bone thickness nearest to the emitter element En. V2, the speed of sound in the skull bone, is not necessarily uniform across the skull. For example, it may vary between thicker and thinner areas of the skull bone.

In preferred embodiments, the first estimation of Wn is given with a pre-determined selected V2 value (e.g., V2=2800 m/s, or 2600 m/s). Then, an iterated estimation of Wn is adjusted based on the value of first estimation of Wn. For example, is Wn is corrected with assuming V2 higher in thinner skull areas and lower at thicker skull areas.

In addition to the phase corrections, amplitude corrections can be determined from the reflected test signals from individual emitters.

In addition, to maximize the transmitted intensity, a step of frequency-test scan is added to be performed prior to treatment application PROC-2, or prior to the SCAN procedure. In the frequency-test scan, the goal is to find the frequency of maximum transmission through the intermediate layer in order to minimize loss of intensity at the focus (due to reflection from the intermediate bone layer) and in order to minimize heat deposition within the intermediate bone layer. In a frequency-scan, for a local region the reflected signal intensity is measured while changing the emitter activation frequency range around a central work frequency, e.g., within 10% deviation from the central work frequency. For example, if the work frequency is chosen to be 500 KHz, a scan of frequency range between 450 KHz and 550 KHz is performed. Minimum local of reflected signal intensity indicates maximum local transmission at the associated frequency. In preferred embodiments, the frequency scan is performed for the array activation as a whole (rather than for local regions).

### BRIEF DESCRIPTION OF THE DRAWINGS

A presently preferred embodiment of the invention will be described in detail, in conjunction with the accompanying drawings, in which:

FIG. 1 is a schematic illustration of a prior art system.

FIG. 2A and FIG. 2B are schematic illustrations of prior art systems.

FIGS. 3A, 3B, 3C and 3D are schematic illustrations of embodiments of focusing transducer arrays, showing, respectively, a spherically-curved transducer array, a flat transducer array with interchangeable lens, a phased-array transducer, and a generic focusing module.

FIG. 3E is a schematic illustration of a two-dimensional transducer array.

FIG. 4A is a schematic illustration of a phased-array transducer causing only steering.

FIG. 4B is a schematic illustration of a phased-array transducer causing steering and focusing at the same time.

FIG. 5 illustrates selected functional components of the transducer array according to embodiments.

FIG. 6A is a schematic illustration of a system when operated in a uniform medium, according to embodiments.

FIG. 6B is a schematic illustration of a system for intra-cranial focusing with skull aberration adjustment, according to a preferred embodiment.

FIG. 6C is a schematic illustration of a system when operated for intra-cranial focusing after aberration adjustment, according to embodiments.

FIG. 7 is a schematic illustration of a system for intra-cranial focusing with skull aberration adjustment, according to a preferred embodiment.

FIG. 8 is a schematic illustration of a two-dimensional array of emitter elements and receiver elements according to a preferred embodiment.

FIG. 9 illustrates a system architecture for intra-cranial focusing with skull aberration adjustment according to a preferred embodiment.

FIGS. 10A and 10B are schematic illustrations of aspects of the disclosed echo detection mechanism according to embodiments.

FIG. 10C is a table of the velocity of sound through various media.

FIG. 11 is a schematic diagram of a switching control module according to embodiments.

FIGS. 12, 13 and 14 are schematic illustrations of preferred selection of receiver sensors association with emitter sensors according to various embodiments.

FIG. 15 illustrates some basic principles of signal detection from selected surface boundaries.

FIG. 16 is a schematic illustration of preferred selection of receiver sensors association with emitter sensors according to embodiments.

FIGS. 17A and 17B shows schematic representations of a emitter transducer array.

FIG. 18 is a schematic illustration of preferred selection of receiver sensors association with emitter sensors according to embodiments.

FIG. 19 illustrates a 2D transducer array arrangement according to embodiments.

FIGS. 20A, 20B, 21, 22, 23A, 23B and 23C present the planning and data of a numerical simulation realization example of the present invention.

FIGS. 24A, 24B illustrate a preferred selection of receiver sensors association with emitter sensors according to an alternative preferred embodiment of the invention.

FIGS. 25A, 25B illustrate a numerical simulation realization of the alternative embodiment of FIGS. 24A and 24B.

### DETAILED DESCRIPTION

The invention is herein described, by way of example only, with reference to the accompanying drawings. With specific reference now to the drawings in detail, it is stressed that the particulars shown are by way of example and for purposes of illustrative discussion of the preferred embodiments of the exemplary system only and are presented in the cause of providing what is believed to be a useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how several forms of the invention may be embodied in practice and how to make and use the embodiments.

For brevity, some explicit combinations of various features are not explicitly illustrated in the figures and/or described. It is now disclosed that any combination of the method or device features disclosed herein can be combined in any manner—including any combination of features—any combination of features can be included in any embodiment and/or omitted from any embodiments.

For ease of reference the following numbers in the figures are meant to refer to as follows
100—focusing system
110—focusing transducer.
111—focus peak, also is the focal center of the focusing transducer in uniform medium 99.
112—emitter array component of the focusing transducer.
113—focusing transducer surface.
114—delay module, creating and outputting the time delay set {F(n)} associated with array elements {E(n)}
115—base-signal generator
116—set of input signals arriving to array elements {E(n)}.
117—m
118—coupling medium
119—focal zone area boundary, preferably defined at half maximum peak intensity.
121—unadjusted focus peak location in the presence an intervening second-tissue intermediate-layer.
123—a first example path of ultrasound in the presence an intervening second-tissue intermediate-layer.
124—a second example path of ultrasound in the presence an intervening second-tissue intermediate-layer.
129—unadjusted focal zone area boundary in the presence an intervening second-tissue intermediate-layer, preferably defined at half maximum peak intensity.
131—adjusted focus peak location in the presence an intervening second-tissue intermediate-layer.
139—adjusted focal zone area boundary in the presence an intervening second-tissue intermediate-layer, preferably defined at half maximum peak intensity.
140—signal generator module.
141n—representative emitter element
145—emitter mux.
146—base signal.
150—intermediate-layer of intervening second-tissue (e.g., skull bone layer)
151—first boundary surfaces of intermediate layer 150.
152—second boundary surfaces of intermediate layer 150.
155—incident test signal.
156—reflected signal from boundary surface 151.
157—reflected signal from boundary surface 152.
160—control and computation module.
161n—representative receiver sensor.
165—positioning module.
170—receiver control and signal analysis module.
172—time shift determiner module.
175—receiver mux.
212—representative 2D array
255—emitter/receiver switching controller.
310—central axis of the emitter transducer traversing the focus peak 111 in uniform medium
311—normal to the outer surface of the intermediate layer (e.g., skull bone) at the crossing point of ray 370.
341 or 141n—a representative emitter.
342—a representative emitter on the other side of the central axis 310 of the transducer.
361 or 161n—receiver to which reflected ray 371 arrives.
361x—receiver sensor near to the left of emitter.
362—receiver to which reflected ray arrives from emitter 342.
370—ultrasound propagation “ray” from emitter 341 as would be in selected uniform medium 99 traversing the focus peak 111.
371—ultrasound propagation “ray” reflected from outer surface 151 of the intermediate layer (e.g., skull bone) of incident ultrasound ray 370.
372—ultrasound propagation “ray” reflected from inner surface 151 of the intermediate layer (e.g., skull bone) of incident ultrasound ray 370.
375—ultrasound propagation “ray” from emitter 342 as would be in selected uniform medium 99 traversing the focus peak 111.
450a—a first orientation of skull bone layer 150
450b—a second orientation of skull bone layer 150
461—receiver to which reflected ray arrives when emitted from emitter 341 and reflected from an alternative tilt of skull bone layer.
561—receiver to which reflected ray 571 arrives at peak intensity.
562—receiver to which reflected ray 572 arrives at peak intensity.
563—intensity distribution of reflected ray 371 at the receiver array.
564—intensity distribution of reflected ray 372 at the receiver array.
571—ultrasound propagation “ray” reflected from outer surface of the intermediate layer (e.g., skull bone) of incident ultrasound ray 370 according to Snell law.
572—ultrasound propagation “ray” reflected from inner surface of the intermediate layer (e.g., skull bone) of incident ultrasound ray 370 according to Snell law.
99—matching medium between the emitter module 110 and the cranial skin surface.
98—intracranial brain tissue medium.

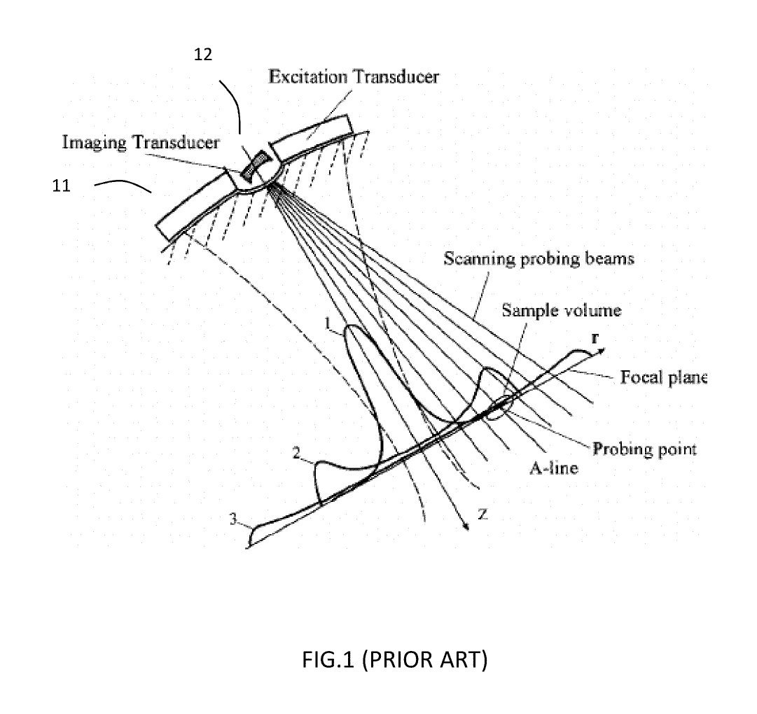

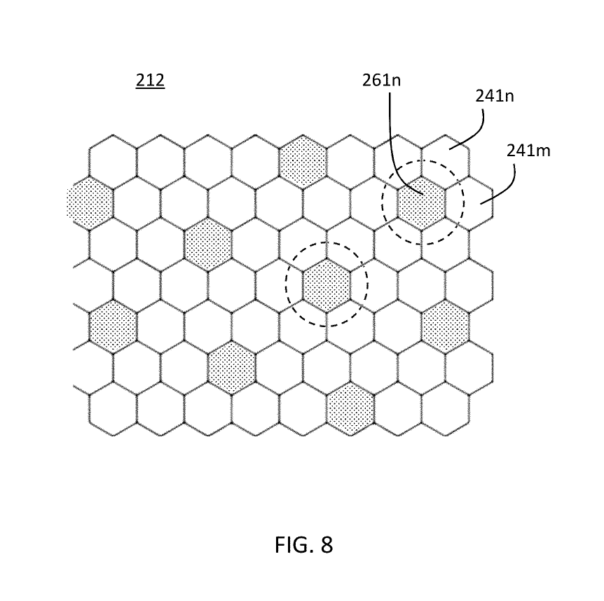

It helps to highlight from the outset certain distinguishing feature of the present invention preferred embodiments in comparison with prior art. FIG. 1 illustrates a prior art embodiment where the focusing emitter module 11 is complemented by a receiver module 12 which is significantly distanced in space from large portion of the emitter module. It will be argued that such geometric combination of emitter and receiver module is less than optimal embodiment for realization of the present invention. In contrast, as will be further elaborated below, preferred embodiments of the present invention comprise a distributed array of receiver array elements which is roughly paralleling in space the distribution of emitter array element, as exemplified in FIG. 8.

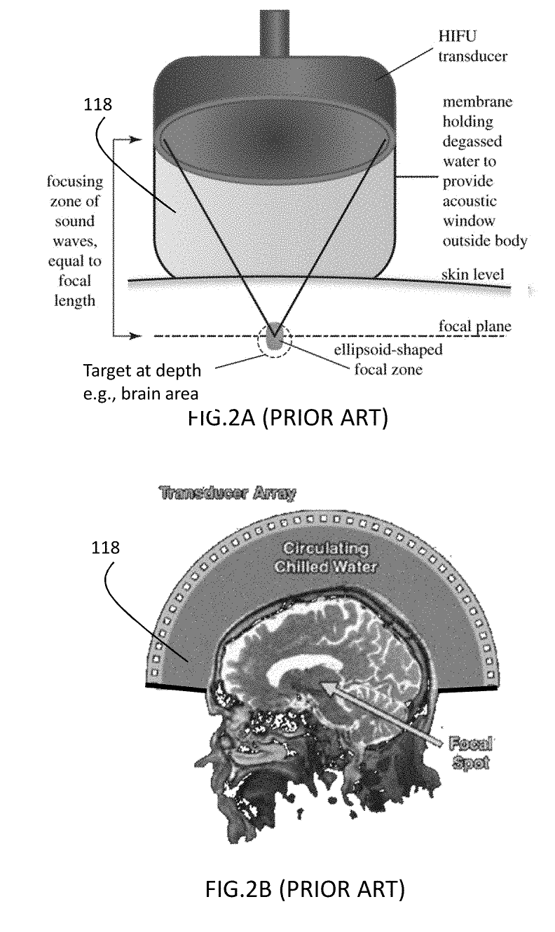

As illustrated in FIGS. 2A and 2B, it is well known in the art that a proper coupling medium 118 needs to fill the gap between the transducer surface 113 and skin surface, in order to reduce ultrasound reflection from the entry interface into the subject body. In preferred embodiments, the coupling medium is deformable, such as a liquid or gel, in order to be able to conform to the skin contour. In preferred embodiments, the coupling medium has speed of sound similar to the skin tissue. FIG. 2B illustrates the art where the coupling medium is a liquid such as water which comes in direct contact with the skin. FIG. 2A illustrates the art where the coupling medium is a liquid such as water which is encased within a retaining membrane. Either one of these methods can be utilized in the complete realization of the present invention. But, since they do not comprise an inventive feature, for the sake of clarity of illustration we shall not explicitly indicated them in the illustration and further discussion of the present invention.

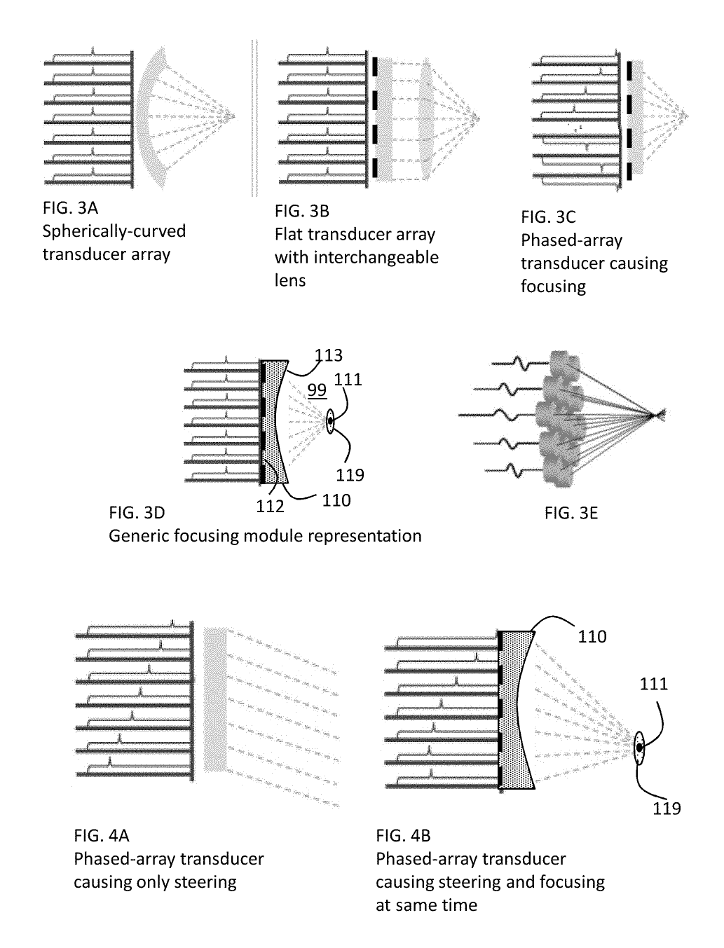

FIGS. 3A, 3B, 3C and 3D illustrate schematic embodiments of focusing transducer emitter arrays {E(n)}, representing alternative preferred embodiments realizations of the basic transducer array as is known in the professional literature to achieve focusing of ultrasound. It is not meant to be limiting, but to the contrary to exemplify the variety of technical possibilities and combinations for the basic transducer array arrangement which is not core to the inventive step of the present invention.

FIG. 3A illustrates a spherical arrangement of the emitter array {E(n)}, where in a uniform medium the focal peak is generated at the sphere center if all the emitter element are activated in uniform phase delay, i.e., {f(n)=f0} where f0 is a constant.

FIG. 3B illustrates a flat transducer emitter array {E(n)} complemented by an acoustic lens. Thereby, in a uniform medium focal peak is generated at the focus center determined by the acoustic lens if all the emitter elements are activated in uniform phase delay, i.e., {f(n)=f0} where f0 is a constant.

FIG. 3C illustrates a flat transducer emitter array {E(n)} with variable relative phase delay between the array element. Thereby, in a uniform medium focal peak is generated at the focus center determined by the set of time delays {f(n)} selected such that the signals emitted from each associated emitter E(n) is a arriving in-phase to desired focal point to create a constructive interference.

Since any of the above and also combination of the above transducer array models can be used to realize the present invention, we use a generic representation, illustrated in FIG. 3D, to signify the focusing transducer 110. Also highlighted in FIG. 3D are certain related aspects of the focusing transducer 110, such as the focus peak location 111 which is also the focal center of the focusing transducer in uniform medium 99, the emitter array 112 component of the focusing transducer, focusing transducer surface 113, and the focal zone 119 area boundary, preferably defined at half maximum peak intensity.

FIG. 4E illustrates schematically a two-dimensional (2D) array of emitter elements. This is meant to highlight the fact that the illustrations in other figures herein, although look graphically like lines, stand to represent 2D and/or 3D arrangements of ultrasound transducer arrays. Hence the simplified graphical representation of other figures is only for the purpose of visual simplicity and not meant to be limiting.

FIGS. 4A and 4B illustrate the ability to steer the focus peak location to more than one location in space using appropriate time delay set for the transducer array, as is well known in the art of ultrasound transducer arrays.

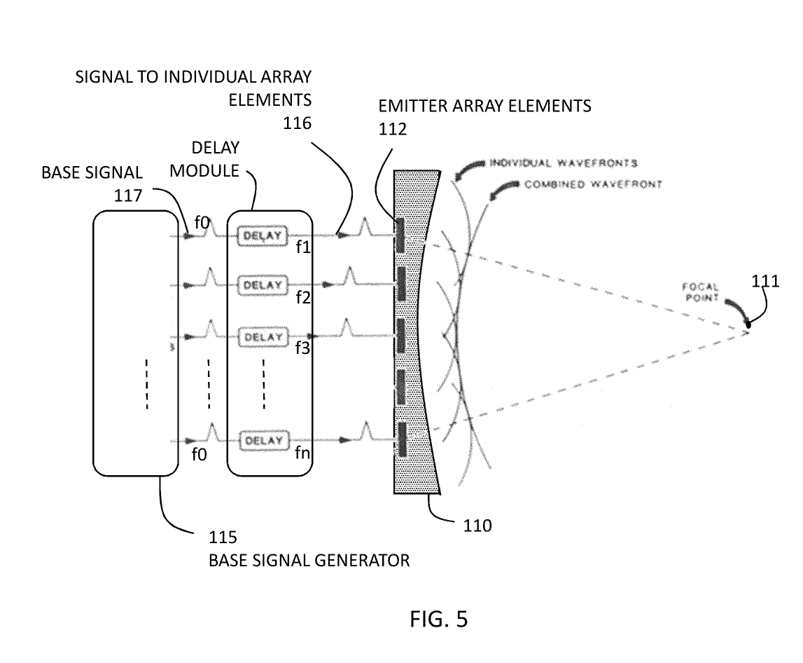

FIG. 5 highlights additional features of a preferred embodiment of the invention system. As is typical in the art of transducer phased array, a base-signal generator 115 produces base signal f0 117. The base-signal f0 is input to and manipulated by a delay-module 114. The delay-module 114 generates a set {F(n)} of time shifted signals 116 each shifted by a time delay F(n) with respect to the input base-signal f0. Optionally, also and amplitude modification of the base-signal amplitude may be generated for each time shifter signal {f(n)}. Optimal focusing is obtained when all signals of the set {f(n)} arrive in-phase to a particular focus peak point 111 at which they have maximum constructive interference.

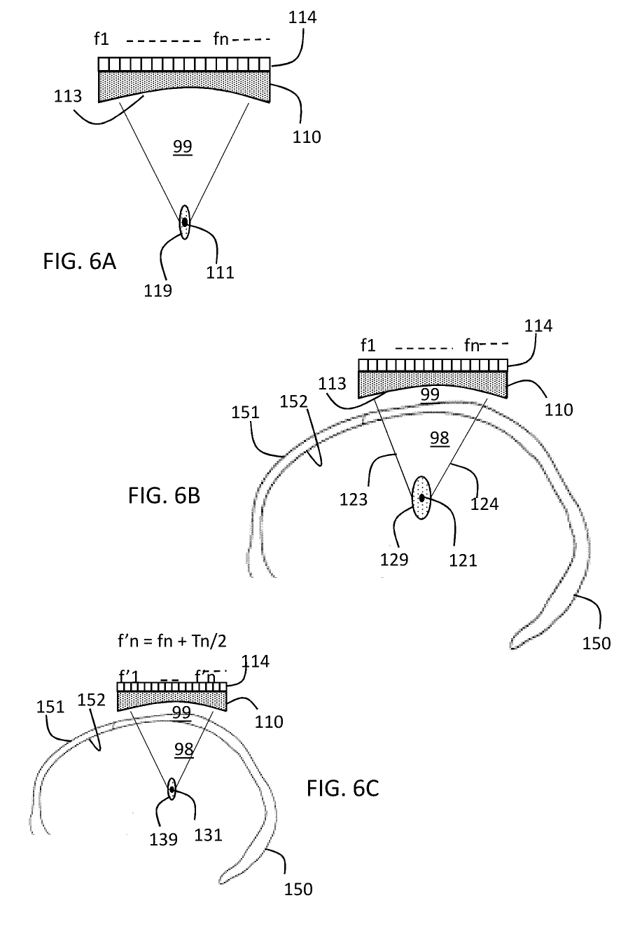

FIGS. 6A, 6B, 6C illustrate the principle problem and goal of the present invention. FIG. 6A illustrates the focus of ultrasound created by transducer 110 when driven by signal delay set {F0(n)}, where the ultrasound waves propagate in uniform medium 99 throughout the path from the transducer surface to the focus peak 111 with focus zone area 119.

As illustrated in FIGS. 6B and 6C, the uniform medium 99 is the matching medium between the transducer 110 and the skull. In contrast, the intracranial skull medium 98 is typically non-uniform. It is preferred, and common in the art, to select a matching medium 99 which is of uniform ultrasound properties and which is characterized by speed of sound which the same (e.g., within less than 10% difference of) as or close to (e.g., within less than 10% difference of) the average speed of sound of the intracranial brain tissue 98.

FIG. 6B illustrates the focus of ultrasound created by transducer 110 when driven by the same signal delay set {F0(n)} as in FIG. 6A, but where the ultrasound waves path pass through an intervening second-tissue intermediate-layer bounded by first 151 and second 152 boundary surfaces within otherwise same uniform medium 99 in the path from the transducer surface to the focus peak 121 with focal zone area 129. Due to the intervening layer, a non-optimal interference is creating the focus peak 129 with focal zone area 129 larger than the uniform medium focal zone 119 and in many cases both shifted focus peak location 121 and more than one focus peak location.

As illustrated in FIG. 6C, the principle goal of the present invention is to drive the focusing transducer 110 with an adjusted signal delay set {F′(n)}={F0(n)+dFn/2}, such that where the ultrasound waves path pass through an intervening second-tissue intermediate-layer bounded by first and second boundary surfaces within otherwise same uniform medium 99 in the path from the transducer surface to the focus peak 131 with focal zone area 139. Due to the adjusted signal delay set {F′(n)}, an improved interference is creating the focus peak 139 with focal zone area 139 smaller than the focal zone 129 created when the focusing transducer 110 with an unadjusted signal delay set {F0(n)}.

The key problem is how to determine the adjustments set {dFn}. The present invention provides a method and system for determine the adjustments set {dFn} and thereby creating an improved focus when the focused ultrasound waves path pass through an intervening second-tissue intermediate-layer bounded by first and second boundary surfaces within otherwise approximately uniform medium 99 in the path from the transducer surface to the focus peak.

We conceptualize the method as a procedure composed of two major stages: (i) scanning procedure “PROC-1”, and (ii) adjusted irradiation application procedure “PROC-2”.

The scanning procedure PROC-1 can be sub-divided into two prominent sub-tasks: (a) physical scanning procedure “SCAN”, and (b) computational analysis “ANALYSIS” from which the key outcome is the determination of a time-set {Tn} from which is determined the adjustments set {dFn}, where each element dFn is a function of corresponding Tn.

The adjusted irradiation application procedure “PROC-2” can be sub-divided into two prominent sub-tasks: (a) Input parameters set-up, and (b) irradiation application process.

In the adjusted irradiation application procedure “PROC-2”, the adjustments set {dFn} from PROC-1, the system is activating the emitters array with the corrected set {F′n} of input parameters phases. The irradiation application process itself is determined by clinical goals (e.g., nerve stimulation, tissue ablation, etc. . . . ).

The innovation is primarily contained in the preparatory scanning process PROC-1 method and apparatus and from it the core outcome is the values of the adjustments set {dFn} of input parameters that is used to define the corrected set {F′n} of phases.

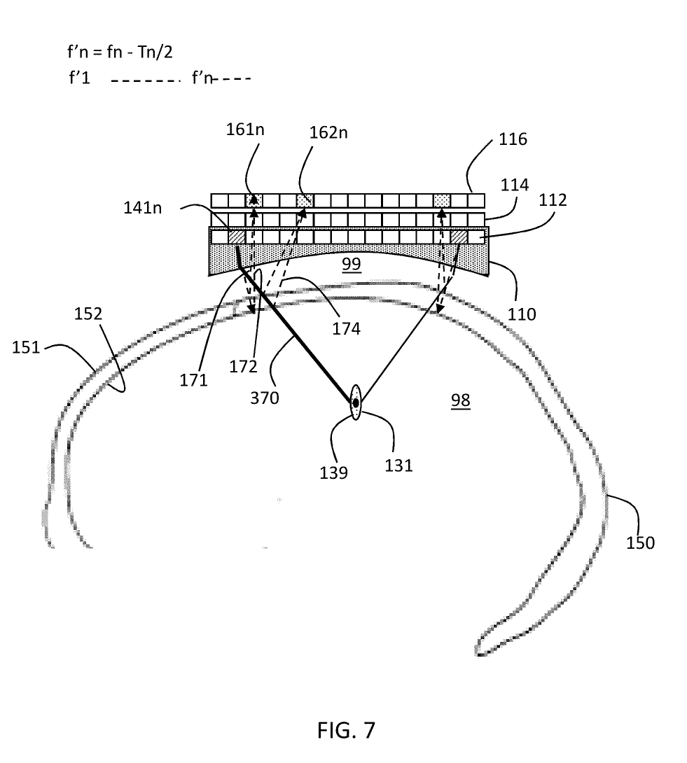

FIG. 7 illustrates a preferred embodiment of the present invention. When a test signal is emitted from an individual emitter 141n selected from the transducer emitters array {E(n)}, there is partial reflection from the first and second boundary surface 151 & 152 of the intermediate tissue layer 150. These reflections can be detected in any one of possible receiver sensors of the receiver array 116. For example, we consider two possible receiver sensors 161n and 162n. In each of the receiver sensors, there will be a time difference “Tn” between the signal reflection from boundary surface 151 and surface 152. The specific time difference Tn is due primarily to the extra path travel within the intermediate layer 150 for the signal reflection from boundary surface 152.

As illustrated in FIG. 7, the reflected signal path 172 arriving to receiver sensor 161n is different from the path of the reflected signal path 174 arriving to receiver sensor 162n. In particular, there is a difference in the length of the partial path through the intermediate layer 150. Consequently, there would be a different value of Tn if the refection is measured with receiver 161n or with receiver 162n.

In the path to the focus peak 111 from the emitter 141n, the sound wave passes once through the intermediate layer 150 width. In contrast, the reflected test signals, 172 and 174, from boundary 152, each passes twice through the intermediate layer 150 width (corresponding to the incident and reflected portions of the path within the layer 150). Therefore, half of the reflected test signals time difference Tn, i.e., (Tn/2), is a good approximation to the time shift contribution of the intermediate layer 150 to the total travel time of the focused beam from the particular emitter 141n to the focus peak location 111. Therefore, in preferred embodiments of the present invention, adjusting the relative time shift by subtracting (Tn/2) from F0(n) substantially eliminates the phase shift contribution of the intermediate layer 150 to the total travel path from the emitter 141n to the focus peak 111. Hence, in preferred embodiments the adjusted delay set {F′(n)}={(F0(n)+dFn)} is then use for irradiating the target tissue using the ultrasound transducer array emitter elements {E(n)} having a corrected-delay {F(n)}={F′(n)} to create a focus peak 131 with an adjusted focal zone 139 of smaller cross section area than if irradiated with unadjusted delay set {F0(n)}.

It remains to be decided which receiver sensor measurement to use for the determination of Tn. Ideally, one would like to have a reflected path which is exactly twice the length of the focused beam path within the intermediate layer 150.

One preferred approximation, as illustrated for the path 172, is a test signal path for which at least the incident portion of the test signal path to be the same as the focused beam path 170 to the focus peak 111. For such an embodiment, fast switching needs to be operated to switch the emitter transducer to receiver mode of operation within the duration of the reflection time. To a good approximation, for ray 370, defined to be the ultrasound propagation “ray” from emitter 341 as would be in the selected uniform medium 99 traversing the focus peak 111, the path of ray 370 through the intermediate layer 150 is very close to the real path of ultrasound ray from emitter 141n to the focus peak 111.

Another preferred embodiment approximation is one for which the same physical transducer array element is used for both test signal emitter and as receiver sensor. Another preferred embodiment approximation is one for which, as illustrated for the path 171, fora given emitter 141n the associated receiver sensor 161n is a neighboring (e.g., nearest neighbor) transducer sensor element 161n. A question in such embodiments is which direction of neighbor to choose as sensor placement relative to the emitter element. For example, should it be one to the left or to the right of the emitter element. The better approximation depends on the relative curvature of the entry boundary 151 of the intermediate layer 150 compared with the curvature of the focus beam at that boundary surface. For example, as illustrated in FIG. 7, if the boundary surface 151 is flatter than the better approximation is to have for emitter element 141n the associated test receiver sensor 161n to the right of it. The opposite choice, of a receiver sensor to the left would be preferred if the boundary surface 151 would be of higher curvature than the focused beam. For most practical purpose applications, the difference between left and right sensors are minuscule, and a preferred embodiment is to have a fixed pre-determined association of a given emitter sensor 141n to s fixed receiver sensor 161n.

There is no need for unique exclusive one-to-one association of a sensor to an emitter. In some preferred embodiments, the number receiver sensors is smaller than the number of emitter elements in {E(n)}. For example, as illustrated in FIG. 8, in a preferred embodiment of 2D array transducer exemplified by the array 212, receiver element 261n can serve for test signal from several nearest neighboring emitter elements marked by the dashed circle, such as 241n and 241m.

In some preferred embodiments, the emitter and receiver sets are physically distinct. In some other preferred embodiments, the emitter and receiver sets are overlapping. For example, referring to FIG. 8, the array of receiver element marked by shaded fill cells may be distinct from the array of emitter elements marked by white fill cells. Alternatively, the shaded fill cells can mark a sub-set of the transducer elements which may be switched between acting as emitter and receiver functionality.

It is preferred to be able to selectively activate individual emitter and/or receiver elements from the physical arrays sets. For example, for test signal and adjustment procedures, one is preferably activating the array emitter elements serially or in sub-sections (i.e., not all together as for focus creation), and respectively receiving and/or analyzing the echo received signals only of the associated receiver sensors. That is relevant both for embodiments where the emitter and receiver sets are physically distinct and for embodiments where they are overlapping. In preferred embodiments, the switching control and activation of the emitter 112 and/or receiver 116 arrays are managed by a controller module 255.

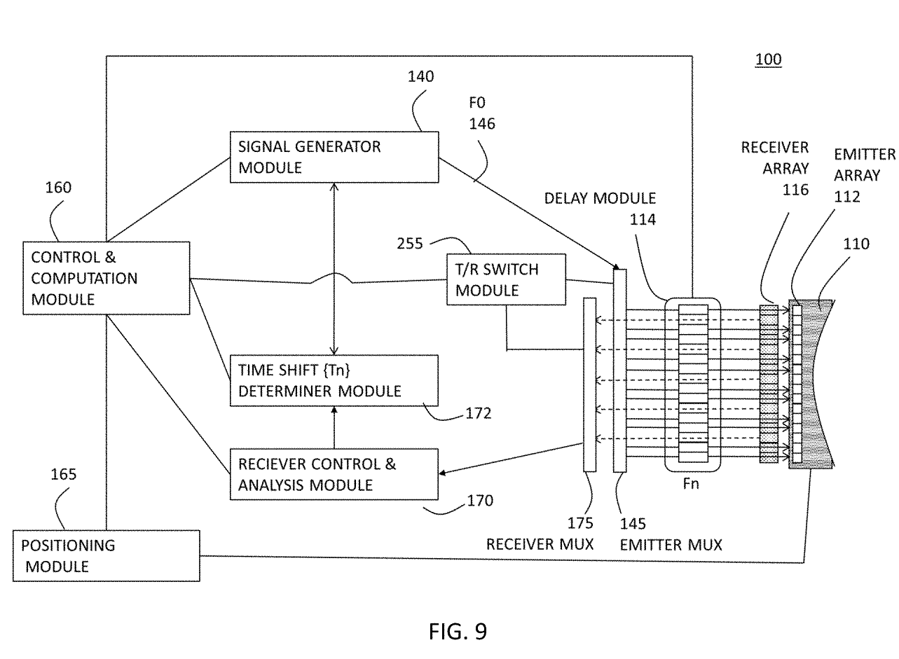

FIG. 9 illustrates a preferred embodiment the invention system for focusing ultrasound into a target tissue when having an intervening second-tissue intermediate-layer 150 bounded by first 151 and second 152 boundary surfaces, using ultrasound sensing, the system comprising:

A focusing transducer 110 comprising an emitter phased array {E(n)} 112 of ultrasound transducer elements for generating an ultrasound focus in the target tissue. At least most transducer elements having means connected thereto for variably setting a delay for that emitter transducer E(n), including a delay module for setting delay set {F(n)} of signals delay to associated emitter elements {E(n)}.

A receiver array 116 comprising a plurality of ultrasound receiver elements {R(n)} associated with emitter elements {E(n)}; The receiver array 116 is connected to and controlled by a receiver control module 170.

A transmit/receive controller “T/R controller” module 255 for directing the activation and switching of individual elements of the emitter and receiver arrays. The T/R module 255 comprising connections to the emitters array 112, to the receiver array 116, to the signal generator module 140 and to the control & computation module 160.

An emitter mux module 145 receives input signal from the signal generator 140, the associated activation delay set {Tn} from control module 160, and the array elements activation directed by T/R module 255. The emitter mux 145 transmit the activation signals to the emitter array 112 elements {E(n)}.

For focusing-mode all, or at least the majority, of the emitter array 112 elements {E(n)} are activated simultaneously, with associated delay set {F(n)} of a common action-signal, to generate a focus peak at a certain location.

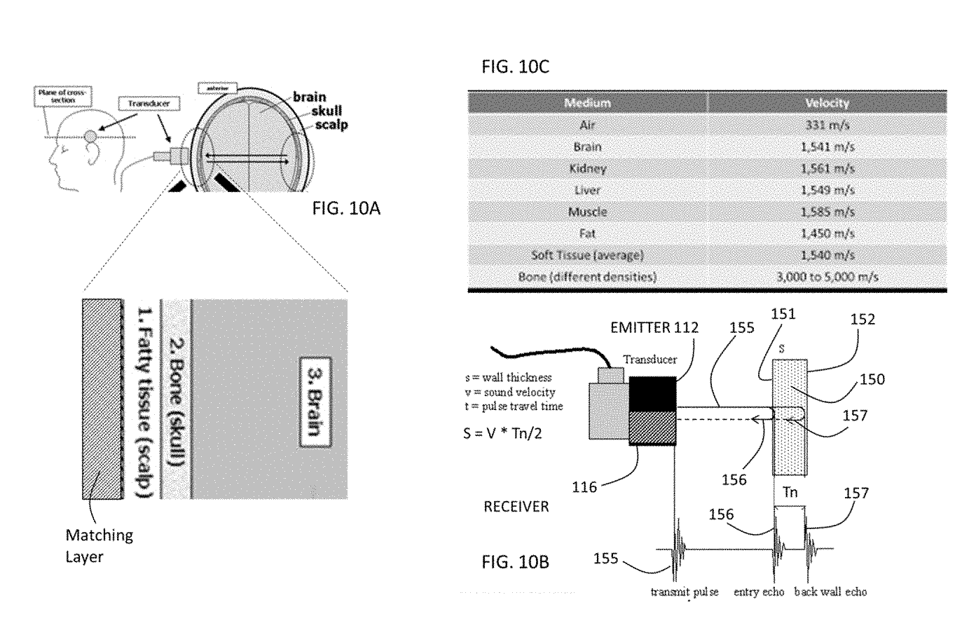

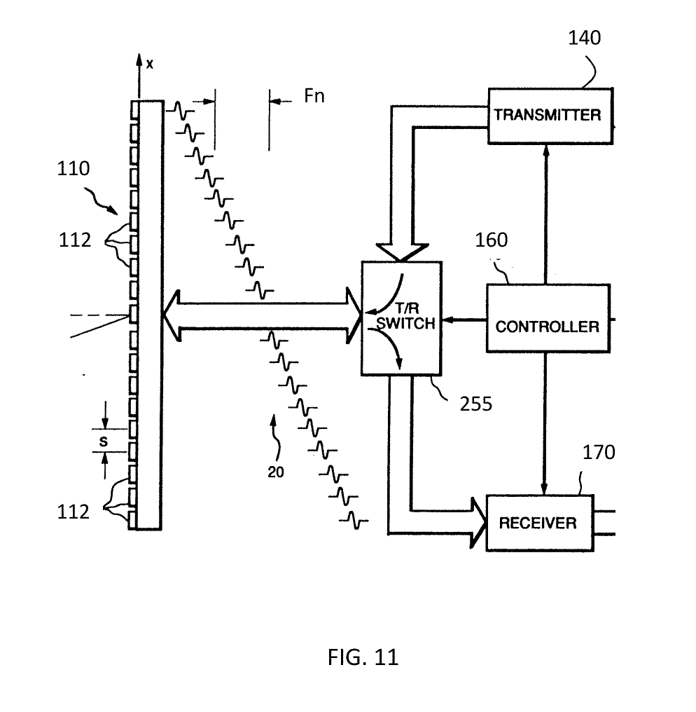

For adjustment-mode a minority, preferably one, of the emitter elements E(n) is activated at a time, preferably with a distinct “test-signal”, which may be preferably different from the action-signal. The test signal reflected echo-signal is received at an associated receiver element of the receiver set 116 and transmitted for analysis to receiver control module 170. It is expected that the received reflected signal would include multiple reflections from various material boundaries such as skin surface, fatty tissue, and the intermediate skull bone layer 150. As highlighted by the table of FIG. 10C, since the biggest difference of speed of sound is cranial ultrasound is between bone tissue and its neighboring tissue, we expect that the reflection from intermediate skull tissue layer 150 boundaries 151 and 152 can be singled out by having the biggest reflection amplitudes compared with other parts of the echo signal. The receiver control module extracts the from the received signal the echo component associated with reflections from the first and second boundary surfaces 151 and 152 of the intermediate layer 150. The time difference between these reflection signals is determined by the time-shift determiner module 172 to create the reflection time difference Tn. Repeating the process serially for multiple, preferably most or preferably all, of the emitter elements, lead to obtaining a reflection time difference {Tn}.

After adjustment mode procedure, the focusing mode is activated with the emitter array 112 driven with an adjusted time-delay set {F′(n)}={F0(n)+dFn} to create an adjusted focus peak.

For effective focus improvement by the invention adjustment procedure. The positioning module 165 maintains the transducer module 110 at a fixed orientation relative to the target tissue for both the adjustment procedure and the focusing mode activation.

FIG. 10A and FIG. 10B highlight some aspects of the extraction of the reflected signals and calculation of the reflection delay time Tn. FIG. 10A highlights the multiple layers of tissue on the path from the transducer emitter outside of the head to the focus peak within the skull. FIG. 10B illustrates schematically the reflection of emitted test-signal 155, first reflected signal 156 from boundary surface 151 is received at the receiver sensor, and later the reflected signal 157 from the boundary surface 152 of the intermediate layer 150 is received at the receiver sensor. The arrival time difference between signal 156 and 157 corresponds to the detected test-signal delay Tn.

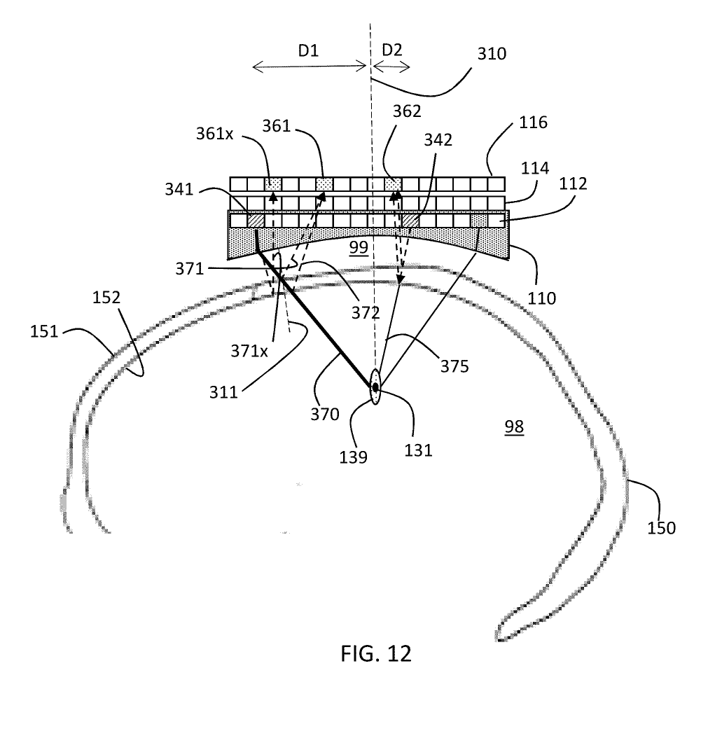

As previously discussed, better focusing adjustment would be obtained if the test signals path through the intermediate tissue layer is better matching (i.e., closer) to the path through the intermediate layer of focusing beam to the focus peak location. As illustrated in FIG. 12, the focusing-axis 310 is defined as the line from the focus peak to geometrical center of the transducer array in uniform medium. For a peripheral-emitter 341 that is further from the focusing-axis 310 than the distance for central-emitter 342 that is closer to the focusing-axis, a better matching of is obtained if the distance D1 between the emitter 341 and associated test-receiver 361 is larger than the distance D2 between the emitter 342 and associated test-receiver 362. For some embodiments, for optimal matching, the difference between D1 and D2 can be a factor of two or more. To a good approximation, for ray 370, defined to be the ultrasound propagation “ray” from emitter 341 as would be in the selected uniform medium 99 traversing the focus peak 111, the path of ray 370 through the intermediate layer 150 is very close to the real path of ultrasound ray from emitter 141n to the focus peak 111. Reflected ultrasound propagation “ray” 371 is reflected from outer surface of the intermediate layer (e.g., skull bone) of incident ultrasound ray 370. Reflected ultrasound propagation “ray” 372 is reflected from inner surface of the intermediate layer (e.g., skull bone) of incident ultrasound ray 370. In FIG. 12 it is illustrated that both reflected rays 371 and 372 signals are measured at the same receiver sensor 361, but this is not meant to be limiting, as will be further elaborated below.

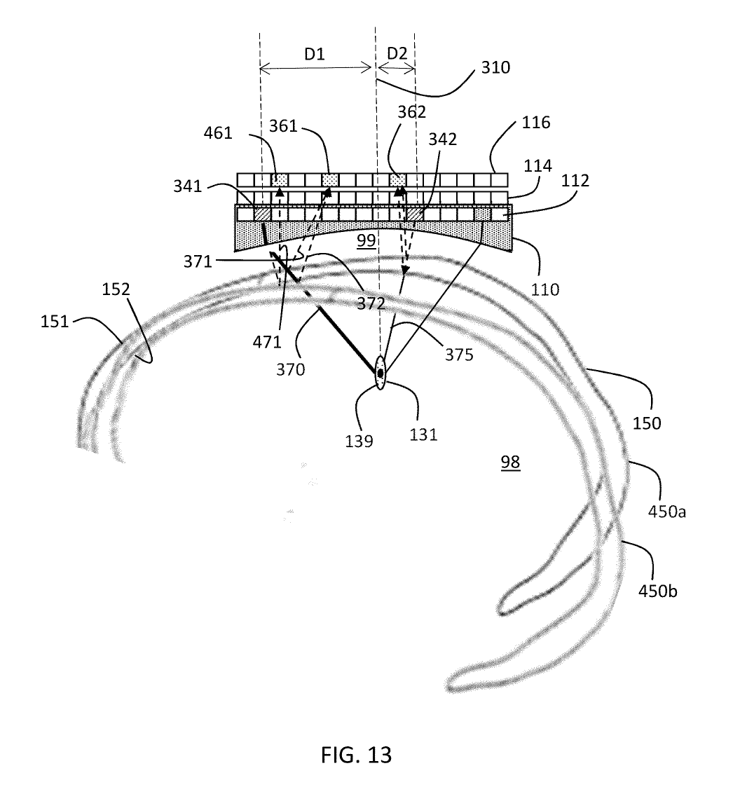

In preferred embodiments, the preferred associated test-receiver for optimal path matching is not fixed for all intermediate layers, but is dependent on the orientation of the intermediate tissue boundary layers relative to the transducer focusing-axis 310. For example, as illustrated in FIG. 13, for a given emitter 341, the preferred associated test-receiver for optimal path matching may depend on the skull orientation with respect to the transducer array focusing-axis 310. For example, for layer orientation 450a the better optimal receiver is 361, while for layer orientation 450b the better optimal receiver is 461. The reason is that the skull orientation changes the intermediate boundary surfaces orientation and hence the ultrasound reflection angles from them. The sensing of the skull orientation with respect to the transducer can also be detected using the ultrasound emitter array 112 and the receiver array 116. i.e., in preferred embodiments of the method of the present invention, prior to the test signal procedure there is a calibration procedure for determining the orientation of the intermediate tissue boundary layers with respect to focusing-axis 310.

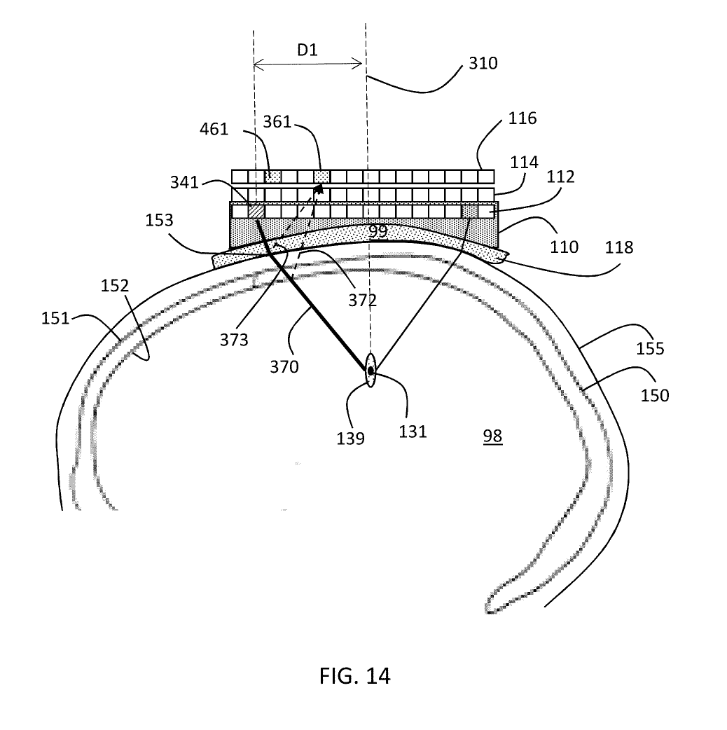

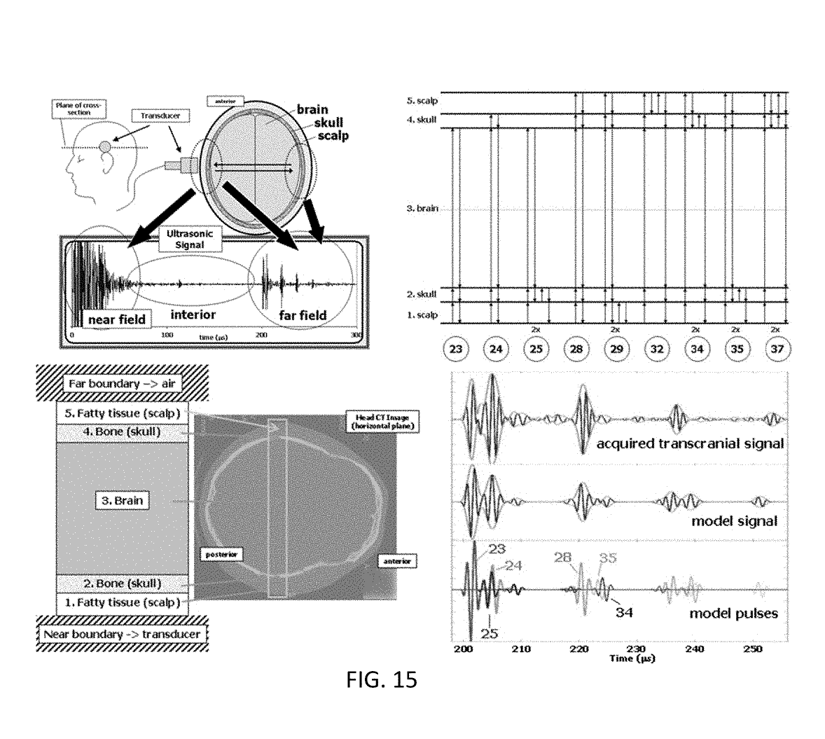

As illustrated in FIG. 14, the aberration effect of the scalp layer (i.e., between the skin surface and the first skull boundary surface 151) is small compared with just the skull bone layer, because (a) the speed of sound in the scalp is very close to the speed of sound of brain tissue and/or the coupling medium 118, and (b) the scalp is relatively uniform in thickness across the surface area of the scalp contact with the device. Hence, the focus of our discussion is on detection and correction of reflections between the first skull boundary entry surface 151 and the second skull bone boundary exit surface 152. Yet, this is not meant to be limiting. The inclusion of the scalp layer 155 is a simple extension of the entry reference to the skin surface 153 entry instead of the bone entry surface 151. Hence, the first reflection delay count would be from the path 173, which is identifiable as known in the art to select among the series of echo received signals.

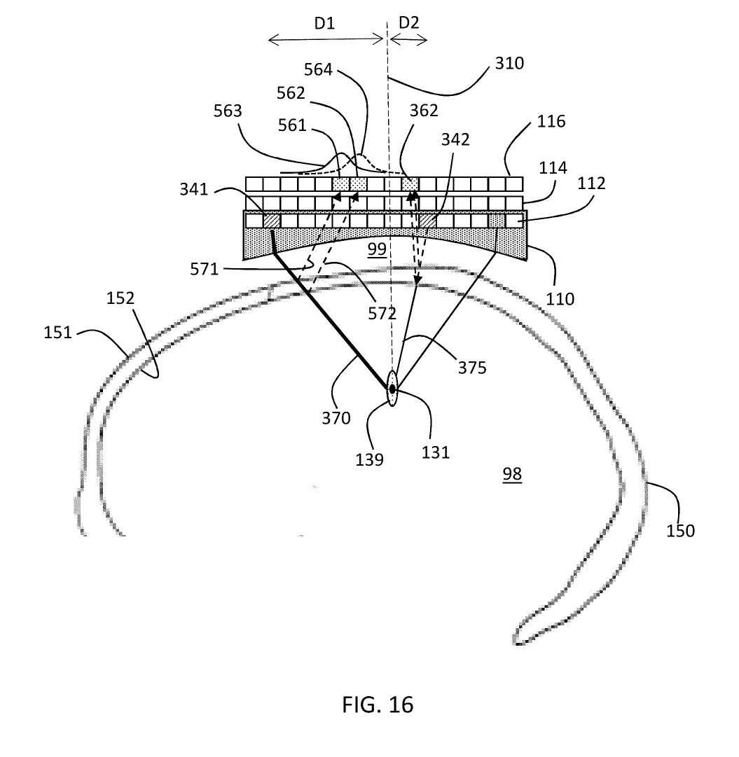

The selection of receiver sensors at which time of reflected beams is determining the time delay correction adjustment. FIG. 16 illustrates a preferred method of receiver sensor selection. As previously discussed, better focusing adjustment would be obtained if the test signals path through the intermediate tissue layer is better matching (i.e., closer) to the path through the intermediate layer of focusing beam to the focus peak location. When a test pulse is emitted from an individual emitter 341, there is a sufficient time gap to distinguish between arrival at the receiver array 116 of the reflection beam signal 571 from first surface (outer surface) 151 and of reflection beam signal 572 from second surface (inner surface) 152 of the intermediate layer 150. The reflected beam signal 571 may be arriving at each receiver of the receiver array 116 at a different time and also at a different intensity with a spatial distribution 563 of intensity which is measured by the receivers array. The reflected beam signal 572 may be arriving at each receiver of the receiver array 116 at a different time and also at a different intensity with a spatial distribution 564 of intensity which is measured by the receivers array. In preferred embodiments, the sensor for timing T1 of the arrival of reflection 571 is timed at receiver 561 at which reflected beam 571 is detected at peak intensity. In preferred embodiments, the sensor for timing T2 the arrival of reflection 572 is timed at receiver 562 at which reflected beam 572 is detected at peak intensity. Repeating the test process for each emitter E(n) we obtain the reflected signals arrival times T1(n) and T2(n).

The time difference between these reflection signals T2(n)−T1(n) is determined by the time-shift determiner module 172 to create the reflection time difference Tn=T2(n)−T1(n). Repeating the process serially for multiple, preferably most or preferably all, of the emitter elements, lead to obtaining a reflection time difference set {Tn}.

After adjustment mode procedure, the focusing mode is activated with the emitter array 112 driven with an adjusted time-delay set {F′(n)}={F0(n)+dFn} to create an adjusted focus peak.

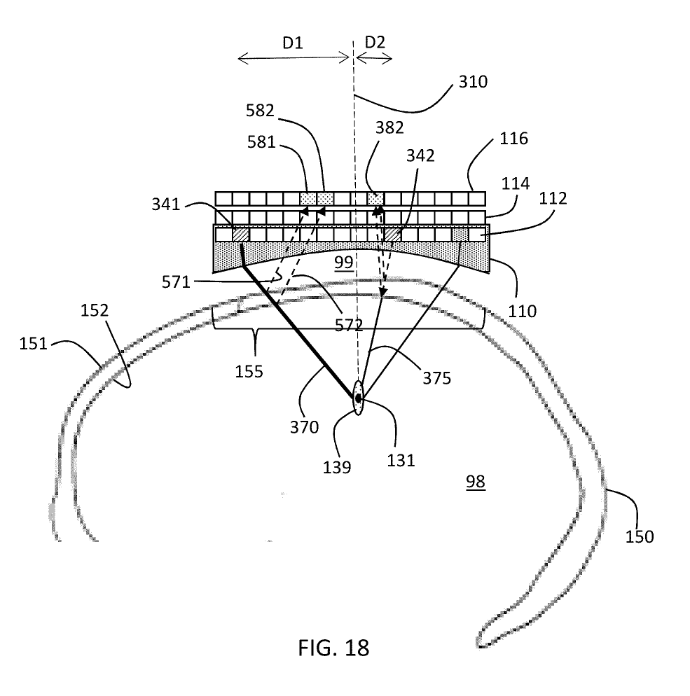

The selection of receiver sensors at which time of reflected beams is determining the time delay correction adjustment. FIG. 18 illustrates a preferred method of receiver sensor selection. As previously discussed, better focusing adjustment would be obtained if the test signals path through the intermediate tissue layer is better matching (i.e., closer) to the path through the intermediate layer of focusing beam to the focus peak location. When a test pulse is emitted from an individual emitter 341, there is a sufficient time gap to distinguish between arrival at the receiver array 116 of the reflection beam signal 571 from first surface (outer surface) 151 and of reflection beam signal 572 from second surface (inner surface) 152 of the intermediate layer 150. The reflected beam signal 571 may be arriving at each receiver of the receiver array 116 at a different time and also at a different intensity with a spatial distribution 563 of intensity which is measured by the receivers array. The reflected beam signal 572 may be arriving at each receiver of the receiver array 116 at a different time and also at a different intensity with a spatial distribution 564 of intensity which is measured by the receivers array.

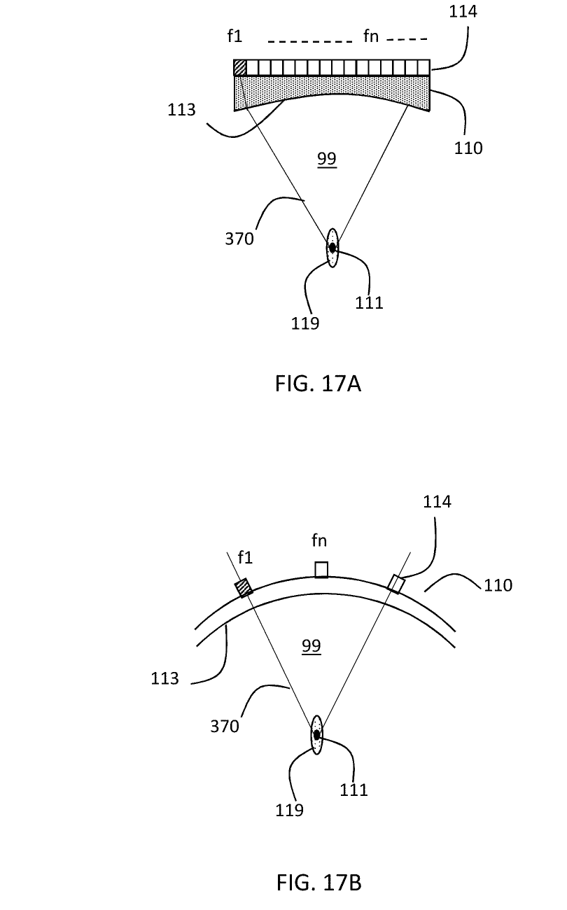

The previously discussed embodiment, with reference to FIG. 16, had the sensor selection based on the reflected intensity. This has a deficit that the test reflection intensity is not changing if the focus location is moved (e.g., by relative phases shifting it left/right of the transducer axis). Hence, there may be some degradation of the level of aberration fixing for different intended focus locations. In contrast, the intended depiction in FIG. 18 is that the selection of receiver sensor at which reflection time is measured is based on a geometrical determination method. First, the skull section 155 of intermediate layer 150 external surface 151 geometry and orientation at the section 155 facing the transducer is measured by ultrasound method. Given the surface section 155, the geometrical ray 370 from each emitter element 341 to the intended focus peak 111 is traced, as shown in FIGS. 17A and 17B.

In preferred embodiments, the sensor for timing T1 of the arrival of reflection 571 is timed at receiver 561 at which reflected beam 571 is detected at peak intensity. In preferred embodiments, the sensor for timing T2 the arrival of reflection 572 is timed at receiver 562 at which reflected beam 572 is detected at peak intensity. Repeating the test process for each emitter E(n) we obtain the reflected signals arrival times T1(n) and T2(n).

The time difference between these reflection signals T2(n)−T1(n) is determined by the time-shift determiner module 172 to create the reflection time difference Tn=T2(n)−T1(n). Repeating the process serially for multiple, preferably most or preferably all, of the emitter elements, lead to obtaining a reflection time difference set {Tn}.

After adjustment mode procedure, the focusing mode is activated with the emitter array 112 driven with an adjusted time-delay set {F′(n)}={F0(n)+dFn} to create an adjusted focus peak.

In addition to phase corrections (i.e., time delays) for individual emitters En, also amplitude corrections are determined in preferred embodiments. For example, for a given emitter test signal, the fractional intensity which is collected at the receiver peak (and optionally including the intensity of the nearest neighbors receivers also) for both the first boundary reflection and second boundary layer reflected echo signal is indicative of the remaining transmitted intensity that reaches the focus peak. Higher reflected fraction RI(n) means lower transmitted intensity fraction “TI(n)” contributing to the focus peak. Hence, in preferred embodiments, the amplitude correction A(n) for each emitter En of the set {En} is a function of TI(n), e.g., proportional to 1/TI(n). For example, in preferred embodiments, in order to obtain more uniform contribution to the focus peak from each emitter En, the intensity emitted from emitter En is set to be [1/TI(n)]*A0, where A0 if the intended average intensity of the transducer emitters array.

In addition, to maximize the transmitted intensity, a step of frequency-test scan is performed prior to treatment application. Typical skull bone thickness ranges between 4 mm to 12 mm. Typical ultrasound treatment frequency ranges between 0.25 MHz to 2 MHz, which at average speed of sound in bone of 3000 m/s translates to a range of wavelength between 1.5 mm to 12 mm. Hence, ¼ wavelengths range in size between 0.375 mm to 3 mm. Due to the ¼ wavelength maximum transmission effect, for a given skull bone sample, for any given desired central frequency W0 for treatment (e.g., 1 MHz, with associated wavelength of 3 mm for bone speed of sound of 3000 m/s) there will be a particular optimal frequency W1 in the neighborhood of W0 for which the transmitted ultrasound total beam intensity across the skull bone layer is maximized. This can respectively be detected as a minimum in the reflected intensity.

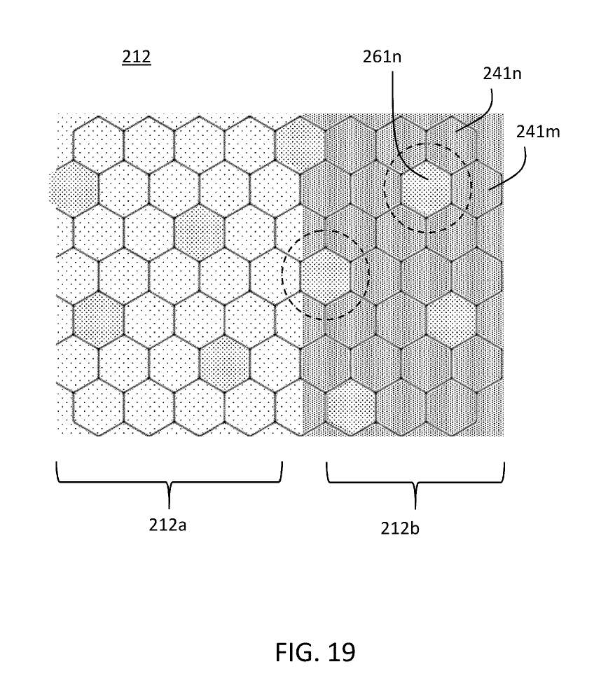

Since the intermediate layer (e.g., skull bone) thickness is non-uniform, it is possible and even likely that, the optimal frequency W1 of maximum transmitted intensity is different for different sub-regions of the surface area 155 under the full transducer area. Therefore, in preferred embodiments, as illustrated in FIG. 19, the 2D surface area of the transducer emitter array 212 is subdivided into a set of two or more sub-sections {212a, 212b, etc . . . }.

The optimal frequency W1 is determined independently for each sub-section. Then, the treatment is delivered with each sub-section driven at its own local optimal frequency, in parallel or serially with other sub-sections of the transducer array.

In all of the above examples, the key in the preparatory SCAN process is to get separate measurements of individual emitters En (or small local group of emitters). In practice, the signals from sufficiently distant emitters do not mix or interfere at the small distance of the SCAN reflection measurements. Therefore, in preferred embodiments, the SCAN process can be performed simultaneously on multiple emitters sub-set of the full array set {En} such that the distance between the array elements is larger than the distance between each array element and the intermediate skull layer 151.

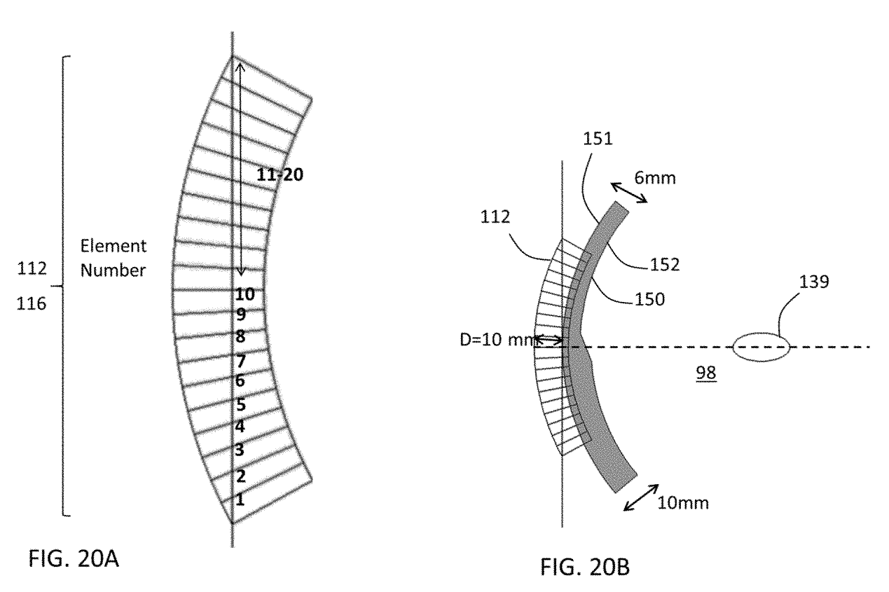

FIGS. 20A, 20B, 21, 22, 23A, 23B and 23C describe a computer simulation which illustrates the method and elements of the apparatus of the present invention. While the simulation was conducted in 2D, its extension to 3D is straight forward to experts of the art. As illustrated in FIG. 20A, the emitters array 112 consists of 20 identical elements emitting ultrasound at frequency of 500 KHz. Each element width is 4 mm, the elements are ordered tightly along an arc of radius of curvature 80 mm. The array elements perform both as emitter and receiver sensors at the same location and thus it is representing also the receivers array 116. As illustrated in FIG. 20B, the intermediate curved layer 150, representing a skull bone element, is bounded by a front/outer boundary surface curve 151 in the shape of a smooth arc and back/inner boundary surface 152 in the shape of a curved step. Thereby, the intermediate payer 150 has asymmetric thickness going from 10 mm width on one side to 6 mm with on the other side. The speed of sound V1 in the uniform medium 98 outside of the intermediate layer 150 is chosen to be V1=1500 m/s. The speed of sound V2 in the inside of the intermediate layer 150 is chosen to be V2=4000 m/s. Hence a difference of 4 mm in passage length through the intermediate layer would cause a relative phase shift of ½ wavelength and result with destructive interference at the intended focus peak location instead of the intended constructive interference needed to create the peak as would be in uniform media 98. Because the curvature of the emitters array 112 and the intermediate layer outer surface 151 are not concentric, the distance between elements En of the array 112 and the intermediate layer surface 151 is not uniform, but it is roughly around 10 mm.

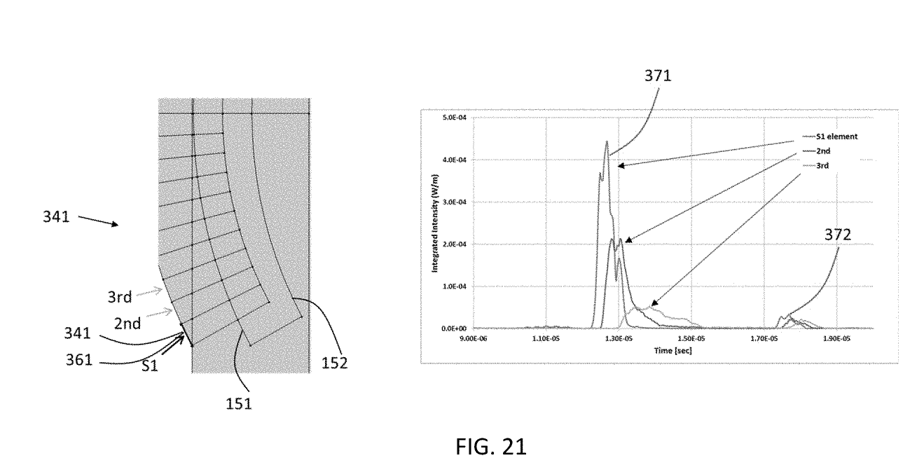

FIG. 21 illustrates the SCAN step performed on one emitter element, labeled “s1”. The signal is excited on S1 element, and measured on the same S1 element and two adjacent neighbors. The first peak at ^{˜}12 micro-sec corresponds to the front reflection 371 from the intermediate layer outer boundary surface. The second peak at ^{˜}17.5 micro-sec corresponds to the back layer reflection 272 from the inner boundary surface. We note that, as expected, there are received echo reflected signals on multiple neighboring receiver elements (such as the nearby ones labeled “2nd” and “3rd” on FIG. 21). Thus, the measurement is a signal-selection process, which in the this simulation is decided by the sensor on which the first reflected echo signal 171 was the strongest intensity. Then the measurement of the second echo reflected signal 172 was chosen to be measured on the same receiver sensor irrespective of its strength. For the emitter element S1 illustrated in FIG. 21, the reflected signals were also measured on the same element E1. This happens to be the case since the array 112 is positioned close to the layer surface 151 and also because the curvature of the array is very close to concentric with the curvature of the intermediate layer.

In preferred embodiments of the present invention the SCAN procedure is the array 112 is positioned close to the layer surface 151. In preferred embodiments of the present invention the curvature of the array is very close to concentric with the curvature of the intermediate layer, typically between 6 cm and 12 cm radius of curvature. Since different areas of the skull have some difference in curvature, and also there is a range of curvature differences between human individuals, the array used in a particular instance can be change-matched to the area of the skull that is irradiated in practice.

The SCAN procedure in the simulation consisted of serially performing the same SCAN step on all the 20 emitters forming the emitters array 112 set {En}.

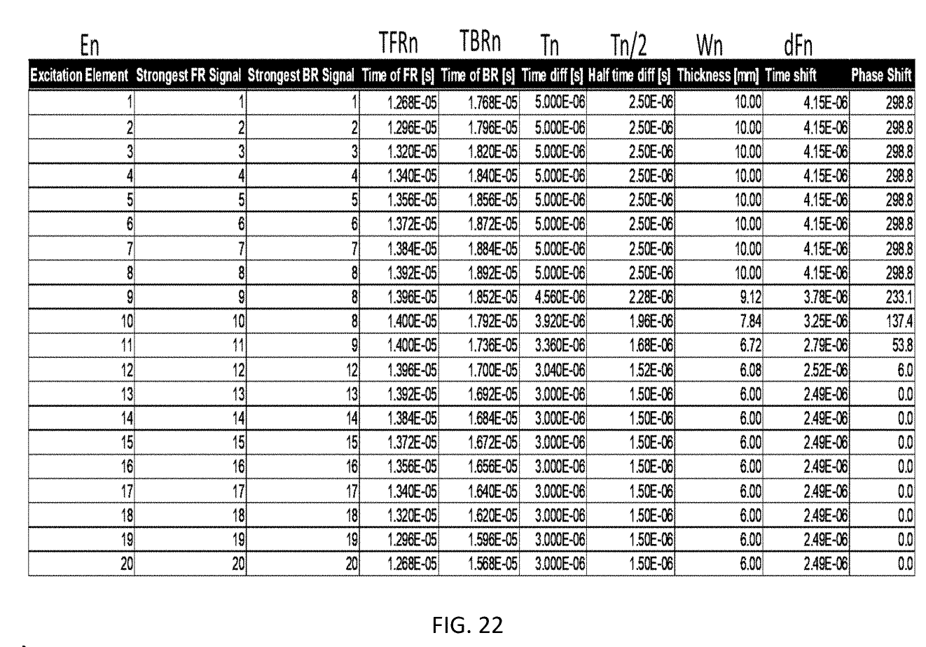

FIG. 22 illustrates the ANALYSIS procedure portion of PROC-1. The first column (#1) numbers the emitters {En} where “n” goes from 1 to 20. Column #2 indicates the emitter on which the first reflected signal from the intermediate layer outer (front) boundary surface 151 surface (which was emitted from that line “n”th emitter) was the largest intensity. Column #3 indicates the emitter on which the reflected signal from the intermediate layer inner (back) boundary surface 152 surface (which was emitted from that line “n”th emitter) was the largest intensity. Column #4 lists the Front-Reflection-Time set {TFRn} of measured time period which took the first reflected signal from the intermediate layer outer (front) boundary surface 151 surface counted from when it was emitted from that line “n”th emitter. Column #5 lists the Back-Reflection-Time set {TBRn} of measured time period which took the first reflected signal from the intermediate layer inner (back) boundary surface 152 surface counted from when it was emitted from that line “n”th emitter. Column #6 lists the resulting set {Tn} of time difference Tn between the two reflected times. This is the main component of the ANALYSIS procedure, from which all else it derived by various estimation approximations. Column #7 lists the resulting estimated layer passage time Tn/2 between the two reflected times. Column #8 lists the resulting estimated layer thickness Wn by using Tn/2 and factoring with the estimated speed of sound V2 in the intermediate layer (at the simplest level used in the present simulation the estimation equation is Wn=V2*Tn/2). Column #9 lists the resulting adjustments set {dFn} of correction factors dFn as function of Tn/2. The function used in the present simulation the estimation equation is dFn=(V2/V1−1)*Tn/2, which for the simulation case with V1=1500 and V2=4000 results in dFn=1.66*Tn/2.

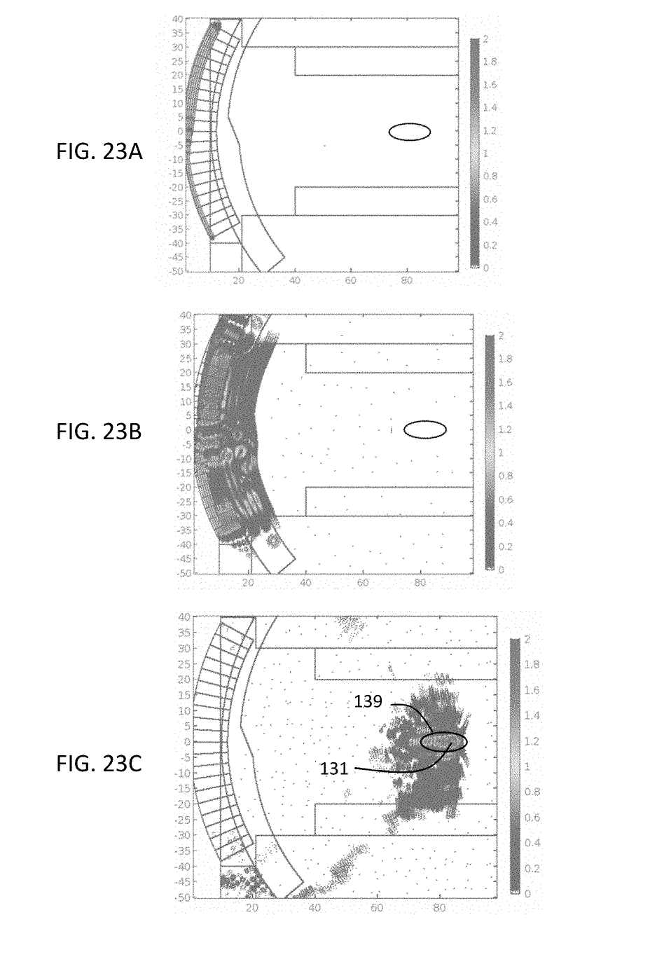

FIGS. 23A, 23B, 23C illustrates the PROC-2 process simulation outcome of performing the adjusted irradiation application procedure. Using the adjustments set {dFn} from PROC-1, the system is activating the emitters array with the corrected set {F′n} of input parameters phases.

FIG. 23A shows a train of several wave crests, a short time after being emitter from the emitters array {En} of 20 emitters and before arriving at the front boundary surface 151 of the intermediate layer 150. There is a visible time delay between the two sides of the array—one facing the narrow side of the intermediate layer 150 and the other side facing the wider side of the intermediate layer 150—creating creates a non-circular wave-front.

FIG. 23B shows a train of several wave crests, a short time after emerging off the back boundary surface 152 of the intermediate layer 150. The wave-front is emerging with a well coordinated circular wave-front between the two sides of the array—one facing the narrow side of the intermediate layer 150 and the other side facing the wider side of the intermediate layer 150, apart from a small and weak misalignment at the central area.

Finally, FIG. 23C shows the waves converging properly to a concentrated peak with the intended peak width 139 and at the intended peak location 131 within few millimeters deviation. Importantly, we note that throughout the process (i.e., SCAN, ANALYSIS, and irradiation), as demonstrated in the simulation, there was no need to recourse to a geometrical reconstruction of the macro shape of the intermediate layer 150. Instead, local measurements where directly translated to local time shifts and adjustment factors.

In the above preferred embodiments examples the PROC-1 SCAN process and the PROC-2 irradiation process where both performed with the emitters array 112 at the same relative position with respect to the intermediate layer 150. But this is not necessarily the case.

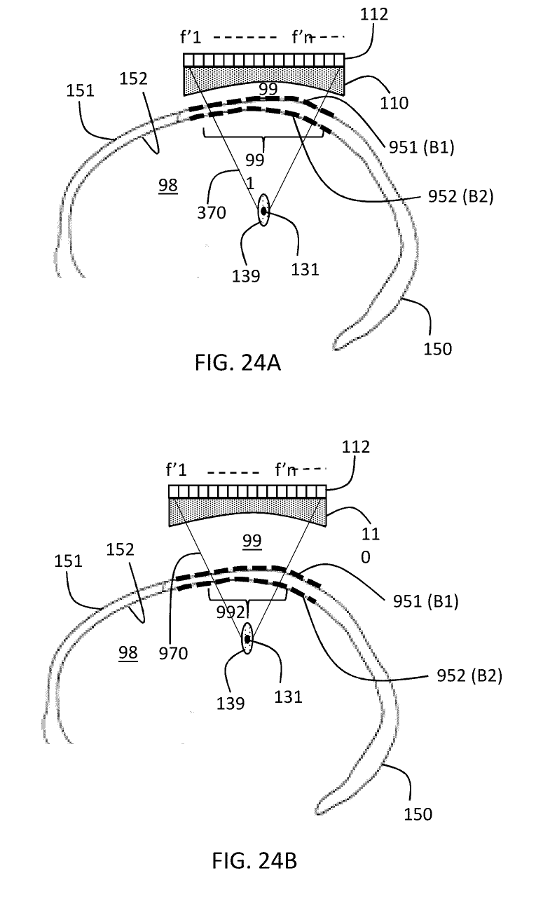

FIGS. 24A, 24B illustrate a preferred embodiment where the PROC-1 SCAN process and the PROC-2 irradiation process where performed with the emitters array 112 at the different relative position with respect to the intermediate layer 150. In particular, as illustrated in FIG. 24B, due to the location of the intended focus relative to the layer 150, it may be preferable to perform the PROC-2 irradiation process with the emitters array 112 relatively at bigger distance from the intermediate layer 150. Yet, as illustrated in FIG. 24A, there may be precision advantages to still perform the PROC-1 SCAN process with the array 112 closer to the intermediate layer 150. The problem is that the path of emitted wave from a given emitter to the intended focus location 131 is not passing through the same intermediate layer section (e.g., skull bone section) in both cases. For example, as illustrated for the emitter E1, the path 370 in FIG. 24A from emitter E1 to the intended focus 131 is passing through a different location in the intermediate layer 150 from the path 970 in FIG. 24B from emitter E1 to the intended focus 131.

In preferred embodiments, in order to enable preferred embodiment where the PROC-1 SCAN process and the PROC-2 irradiation process where performed with the emitters array 112 at the different relative position with respect to the intermediate layer 150, we introduce in PROC-1 an optional supplemental ANALYSIS step—labeled LAYER RECONSTRUCTION—and introduce in PROC-2 a supplemental irradiation preparation optional step—labeled PATH ANALYSIS. The key additional step is the LAYER RECONSTRUCTION sub-process at the end of the ANALYSIS process.

The LAYER RECONSTRUCTION outcome is a geometrical reconstruction of a portion of the intermediate layer which is facing the emitters array 112 during the SCAN process. For concreteness, we define the intermediate layer geometry by estimation of outer boundary surface 151 shape function B1(x,y,z) and the inner layer 152 shape function B2(x,y,z). In the prior-art, these were deduced from external scans by MRI or CT. In contrast, in the present invention the geometrical functions reconstruction is derived from the set of parameters obtained in the invention SCAN process and ANALYSIS. As indicated in FIG. 24A, the SCAN process ANALYSIS provides in formation only on a limited section of the intermediate layer (e.g., skull bone) and therefore only for sub-section 951 there is a reconstructed shape function B1 of estimated outer layer and for sub-section 952 there is a reconstructed shape function B2 of estimated inner layer.

As known in prior art, there are multiple methods of determining the adjustments set {dFn} if there is a given geometry of the intermediate layer (e.g., skull bone layer). Hence, there is no need for us to elaborate how the irradiation process is done once the LAYER RECONSTRUCTION outcome is given.

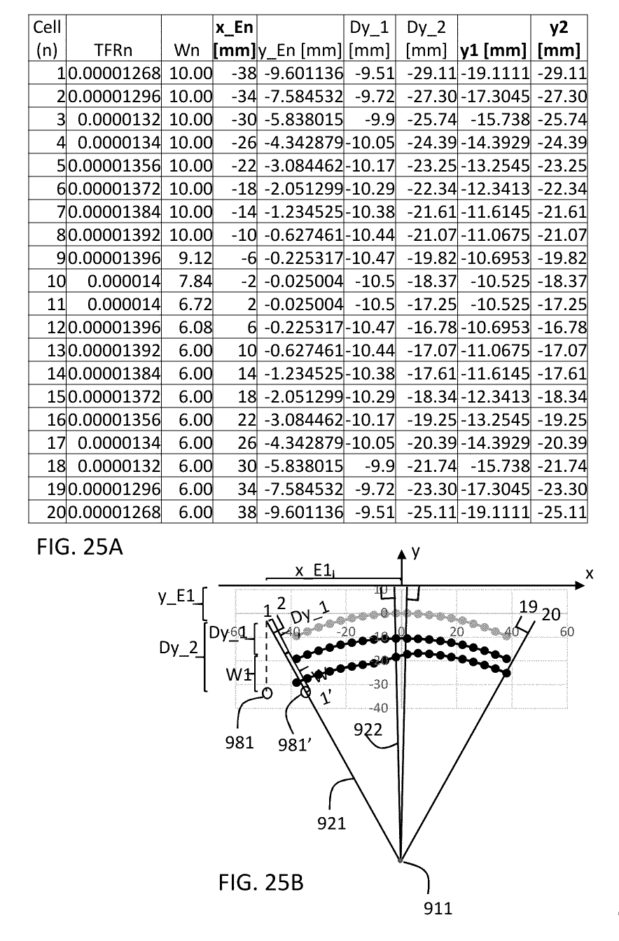

FIGS. 25A and 25B illustrate the LAYER RECONSTRUCTION process, using the ANALYSIS data of the simulation example discussed before. The LAYER RECONSTRUCTION process comprises the following set of input parameters:
i. the known geometrical shape of the emitters array 112; and supplemented by data from the ultrasound SCAN:
ii. the Front-Reflection-Time set {TFRn} of measured time period which took the first reflected signal from the intermediate layer outer (front) boundary surface 151 surface counted from when it was emitted from that line “n”th emitter;
iii. at least one of (A) the Back-Reflection-Time set {TBRn} of measured time period which took the first reflected signal from the intermediate layer inner (back) boundary surface 152 surface counted from when it was emitted from that line “n”th emitter, or (B) the resulting estimated layer passage time {Tn/2} between the two reflected times, from which we can the resulting estimated layer thickness {Wn} by using Tn/2 and factoring with the estimated speed of sound V2 in the intermediate layer (at the simplest level used in the present simulation the estimation equation is Wn=V2*Tn/2).

The usage of these input parameters to reconstruct the intermediate layer outer boundary function B1 and inner boundary function B2 is a matter of approximation. Any approximation procedure gives a mathematically non-identical outcome. Moreover, small deviation from precise functional reconstruction of the true intermediate layer geometry may be of little significance to the intended clinical outcome of the irradiation procedure. Therefore, it is not the particular functional approximation which is the essence of the inventions, but it is the set of above noted input parameters obtained from the SCAN process and ANALYSIS. To highlight this statement, the illustrated approximation procedure in FIGS. 25A and 25B from the simulation data is intentionally simple yet NOT the best mathematical approximation to approximate the intermediate layer geometry.

The table illustrated in FIG. 25A lists the following derived information: (i) “x_En”, the “x” location of each emitter is taken to be the center of the emitter. (ii) “y_En”, the “y” location of the center of each emitter, which in the case of the simulation were on a section of circular arc with radius of curvature of 80 mm. (iii) “Wn”, estimated layer thickness. (iv) “TFRn”, of measured time period which took the first reflected signal from the intermediate layer outer (front) boundary surface 151 surface. The following estimated geometrical locations: (v) “Dy_1” the estimated y-axis distance of the outer (front) boundary surface 151 surface for the same x-axis coordinate as the corresponding emitter. In the presented approximation we take this to be equal to ½ the first reflection distance traveled by the sound (which will be discussed below to be a non-optimal approximation), thus Dy_1 is simply computed as Dy_1=(TFRn/2)*V1. (vi) “y1” is the y-axis coordinate of outer (front) boundary surface 151 surface for the same x-axis coordinate as the corresponding emitter, and is then computed simply y1=y_En+Dy_1. (vii) “Dy_2” is the estimated y-axis distance of the inner (back) boundary surface 152 surface from the outer boundary surface 151, for the same x-axis coordinate as the corresponding emitter. In the presented table this distance was approximated as equal to Wn (which will be discussed below to be a non-optimal approximation), and is then computed simply as Dy_2=Dy_1+Wn. (viii) “y2” is the y-axis coordinate of inner (back) boundary surface 152 surface for the same x-axis coordinate as the corresponding emitter, is then simply y2=y_En+Dy_2 (which will be discussed below to be a non-optimal approximation).

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art.

What is claimed is:

1. A method for generating an adjusted ultrasound focus of onto a target tissue when having an intervening second-tissue intermediate-layer bounded by first and second boundary surfaces, using an ultrasound array having a plurality of transducer emitter elements {E(n)}, each transducer element E(n) having a variable delay F(n) associated therewith, including a delay set {F0(n)} used for a uniform-space focusing of said ultrasound array, and a plurality of ultrasound receiver elements {R(n)} associated with emitter elements set {E(n)}, said method comprising the steps of:
a. Performing an adjustment procedure comprising:
i. Emitting a test-signal from emitter En with delay F0(n);
ii. Detecting reflected test-signal in test-receiver sensor R(n);
iii. Extracting reflection time difference Tn between the test signal reflection from the intermediate-layer first and second boundary surfaces;
iv. Defining a corrected-delay F′(n) proportional to half the reflection time difference Tn, such that F′(n)=(F0(n)+dFn) where dFn is a function of Tn/2;
b. Irradiating the target tissue with a focusing-signal using the ultrasound transducer array emitter elements {E(n)} having a corrected-delay {F(n)}={F′(n)} associated therewith.

2. A method as claimed in claim 1, wherein the function dFn=(V2/V1−1)*Tn/2, where V2 is an estimated average speed of sound in the intermediate-layer within at least 20% accuracy and V1 is an average speed of sound in the target tissue within at least 10% accuracy.

3. Claim 2, wherein V1=1500 m/s.

4. Claim 2, wherein V2=2800 m/s.

5. A method as claimed in claim 1, wherein the adjusted procedure is further defined by repeating the steps (i) to (iv) for at least two different the emitter elements selected from the set {E(n)}.

6. A method as claimed in claim 1, wherein the adjusted procedure is further defined by repeating the steps (i) to (iv) for the majority of the emitter elements {E(n)}.

7. A method as claimed in claim 1, wherein the test-signal ultrasound principle frequency is of higher frequency than the focusing-signal ultrasound principle frequency.

8. A method as claimed in claim 3, wherein, the focusing-axis is defined as the line from the focus peak to geometrical center of the transducer array in uniform medium, the distance between the emitter and associated test-receiver is at least twice as large for a peripheral-emitter that is further from the focusing-axis than the distance for central-emitter that is closer to the focusing-axis.

9. The method as claimed in claim 1, wherein reflection time difference Tn is determined as equal to the difference T2−T1 between: (a) the time T1 of arrival of the test signal reflection from the intermediate-layer first boundary surface at the receiver sensor, selected from the receivers array, at which that reflection is the highest cumulated intensity and (b) the time T2 of arrival of the test signal reflection from the intermediate-layer second boundary surface at the receiver sensor, selected from the receivers array, at which that reflection is the highest cumulated intensity.

10. A system for focusing ultrasound into a target tissue when having an intervening second-tissue intermediate-layer bounded by first and second boundary surfaces, using ultrasound sensing, the system comprising:
a. an emitter phased array {E(n)} of ultrasound transducer elements for generating an ultrasound focus in the target tissue, at least most transducer elements;
b. a delay module for creating a delay set {F(n)} having means connected thereto for variably transmitting a delay signal for that emitter transducer elements {E(n)}, including a delay set {F0(n)} used for a uniform-space focusing of said ultrasound array;
c. a plurality of ultrasound receiver elements {R(n)} associated with emitter elements {E(n)};
d. a control system comprising means for selectively activating sub-sets of emitter array elements {E(n)} (e.g., individual array elements E(n));
e. a detection subsystem, in communication with the emitter phased array {E(n)} and the receiver elements {R(n)} and in communication with the control system;
i. the detection subsystem further comprising means for identifying and processing ultrasound echo test signals reflected by said intermediate-layer first and second boundary surfaces—using a selectively activated emitter element E(n) of the phased array and a selected associated receiver element R(n);
ii. the detection system further comprising a computation module configured to: receive data associated with the echo signal associated with the activated emitter element E(n), based at least in part on the data compute the reflection time difference Tn between the test signal reflection from the intermediate-layer first and second boundary surfaces, compute a corrected-delay F′(n) proportional to half the reflection time difference Tn, such that F′(n)=(F0(n)+dFn) where dFn is a function of Tn/2;
iii. the detection system further comprising means for serially performing a scan of such echo signals from a plurality of selected array emitter elements (e.g., majority of array emitter elements), thereby generating a corrected delay set {F′(n)};
f. the control system further comprising means for driving the transducer array elements {E(n)} at the corrected delays {F′(n)}, so as to generate an improved the ultrasound focus compared with the case where the relative phases where {F0(n)}.

11. The system of claim 10, wherein the function dFn=(V2/V1−1)*Tn/2, where V2 is an estimated average speed of sound in the intermediate-layer within at least 20% accuracy and V1 is an average speed of sound in the target tissue within at least 10% accuracy.

12. The system of claim 10, further comprising a positioning module capable of maintaining the position of the emitter phased array with respect to the target tissue.

13. The system of claim 10, wherein a transducer emitter element E(n) can physically serve also as a receiver element R(m), associated with same emitter n (m=n) and/or associated with a different emitter element n′ (m=n′).

14. The system of claim 10, having at least two modes of activation, (i) a focusing beam mode at which the majority of the emitter array elements are simultaneously emitting ultrasound to create a focus peak, and (ii) an adjustment mode at which only a minority of emitter elements are simultaneously activated and at least one receiver element is sensing the reflected signals and transmitting them for analysis to the computation module.

15. The previous claim wherein the minority of emitter array elements is less than 10% of the array elements, or less than 1% of the array elements.

16. The previous claim wherein the minority of emitter array elements is an individual single element.
